(19) United States
(12) Patent Application Publication
(10) Pub. No.: US 2007/0014788 A1
(43) Pub. Date: Jan. 18, 2007

(54) Laminin-5 modulators and uses thereof

(75) Inventors: Ida Mathiasen, Kgs Lyngby (DK); Michael Karl Bauer, Vanlose (DK); Jorn Roland Muller, Virum (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/451,583

(22) Filed: Jun. 12, 2006

Related U.S. Application Data
(63) Continuation of application No. PCT/DK04/00860, filed on Dec. 13, 2004.

(51) Int. Cl. A61K 48/00; A61K 39/395

(52) U.S. Cl. 424/133.1; 514/44

(57) ABSTRACT

Described herein are methods of treating or preventing various diseases and disorders using modulators of laminin-5. Exemplary diseases and disorders are inflammatory bowel disease, including ulcerative colitis and Crohn's disease, as well as polycystic kidney disease and cancers associated with one or more of the aforementioned conditions. Exemplary laminin-5 modulators include monoclonal antibodies against the laminin-5 gamma2 chain and short interfering RNA (siRNA) sequences against nucleotide sequences encoding the laminin-5 gamma2 chain. Described are also combination treatments based on the administration of a laminin-5 modulator and at least one other therapeutic agent.

### CROSS-REFERENCE TO RELATED PATENT APPLICATIONS

This patent application is a continuation of International Patent application PCT/2004/000860 (published as WO 2005/056598), filed Dec. 13, 2004 (and which designates the United States), and further claims the benefit of U.S. Provisional Patent Application 60/529,067, filed Dec. 12, 2003, the entirety of each of which is hereby incorporated by reference.

### FIELD OF THE INVENTION

The present invention relates to methods and compositions for treatment and/or prevention of diseases or disorders associated with altered expression and/or function of laminin-5.

### BACKGROUND OF THE INVENTION

Inflammatory bowel disease (or “IBD”) is a disorder marked by inflammation of the gastrointestinal (“GI”) tract that typically is not caused by other factors such as infections, medications, or malignancy. IBD encompasses at least two related conditions: ulcerative colitis (UC) and Crohn's disease (CD). UC typically affects only the colon and can be treated in some instances by surgically removing the colon (colectomy). Most patients with CD also will require at least one operation in their lifetime. However, CD generally is not currently considered curable. Research in the field has demonstrated that there is an increased risk of developing colorectal cancers in many CD and UC patients. Colorectal cancer is a leading cause of death in the North America and Europe.

For these and other reasons, there remains a need for alternative and improved methods of promoting, enhancing, and/or inducing the treatment and/or prevention of IBD and IBD-related cancers. The invention described herein provides such methods and novel compositions useful in the practice of such methods, as well as methods of inducing, promoting, and/or enhancing the treatment and/or prevention of other diseases and disorders including irritable bowel syndrome, polycystic kidney disease (PKD), and non-IBD-associated cancers. These and other advantages of the invention, as well as additional inventive aspects and features, will be apparent from the description of the invention provided herein.

### SUMMARY OF THE INVENTION

This invention provides various methods of inducing physiological responses associated with the treatment and/or prevention of various diseases and disorders including, for example, IBD, IBD-associated cancers, irritable bowel syndrome (“IBS”), and PKD, caused by, e.g., autosomal polycystic kidney disease (ADPKD), using a modulator of laminin-5. Described herein are also combination methods, i.e., methods of treating the aforementioned disease and disorders, as well as cancer, using a combination of a laminin-5 modulator and a second agent.

Thus, in one embodiment, the invention provides a method of treating inflammatory bowel disease comprising the step of administering to a subject suffering from inflammatory bowel disease a composition comprising an effective amount of a laminin-5 modulator. In another embodiment, the invention provides a method of reducing the risk of inflammatory bowel disease-associated colorectal carcinoma comprising the step of administering to a subject suffering from inflammatory bowel disease a composition comprising an effective amount of a laminin-5 modulator. In one aspect, the inflammatory bowel disease is ulcerative colitis. In a second aspect, the inflammatory bowel disease is Crohn's disease.

The invention also provides a method of treating polycystic kidney disease comprising the step of administering to a subject identified as suffering from polycystic kidney disease a composition comprising an effective amount of a laminin-5 modulator. In another embodiment, the invention provides a method of reducing the progression of polycystic kidney disease comprising the step of administering to a subject identified as suffering from autosomal dominant polycystic kidney disease a composition comprising an effective amount of a laminin-5 modulator.

The invention also provides a method of treating cancer comprising the step of administering to a subject suffering from cancer a composition comprising effective amounts of a laminin-5 modulator and at least one secondary anti-cancer agent.

In a particular aspect, in any of the foregoing methods, the laminin-5 modulator is selected from the group consisting of an antibody directed against laminin-5, a laminin-5 binding protein, an siRNA, an antisense molecule, or a small molecule Ln-5 modulator. IN another aspect, the method of claim 8, wherein the laminin-5 modulator is an antibody directed against the laminin-5 gamma2 chain. In yet another aspect, the laminin-5 modulator is an siRNA directed against a nucleic acid encoding the laminin-5 gamma2 chain.

The invention also provides for the use of a laminin-5 modulator in the preparation of a medicament for treating inflammatory bowel disease. In one aspect, the inflammatory bowel disease is ulcerative colitis. In another aspect, the inflammatory bowel disease is Crohn's disease.

The invention also provides for the use of a laminin-5 modulator in the preparation of a medicament for treating inflammatory bowel disease-associated colorectal carcinoma.

The invention also provides for the use of effective amounts of a laminin-5 modulator and at least one secondary anti-cancer agent in the preparation of a medicament for treating cancer.

The invention also provides for the use of effective amounts of a laminin-5 modulator and at least one secondary anti-cancer agent in the preparation of a medicament for reducing tumor growth.

In a particular aspect, in any of the foregoing uses, the laminin-5 modulator is selected from the group consisting of an antibody directed against laminin-5, a laminin-5 binding protein, an siRNA, an antisense molecule, or a small molecule Ln-5 modulator. In one aspect, the laminin-5 modulator is an antibody directed against the laminin-5 gamma2 chain. In another aspect, the laminin-5 modulator is an siRNA directed against a nucleic acid encoding the laminin-5 gamma2 chain.

These and various additional aspects of the invention are described elsewhere herein.

### EXEMPLARY ASPECTS AND FEATURES OF THE INVENTION

To better illustrate the invention described herein, a nonlimiting list of exemplary aspects and features of the invention is provided here:

1. A method of inducing, promoting, and/or enhancing one or more physiological responses associated with the treatment or prevention of inflammatory bowel disease comprising administering to a patient identified as having or being at significant risk of developing inflammatory bowel disease a composition comprising a laminin-5-associated antibody, a laminin-5 binding protein, or both in an amount sufficient to detectably induce, promote, and/or enhance the one or more physiological responses.

2. A method of reducing the severity, spread, onset, or risk of developing inflammatory bowel disease and/or promoting the treatment thereof comprising administering to a patient identified as having or being at significant risk of developing inflammatory bowel disease a composition comprising a laminin-5-associated antibody, a laminin-5 binding protein, or both in an amount sufficient to detectably reduce the severity, spread, onset, or risk of developing and/or promote the treatment of inflammatory bowel disease in a population of similar patients.

3. The method of aspect 1 or aspect 2, wherein the method comprises administering a therapeutically effective amount of a laminin-5-binding integrin to the patient.

4. The method of aspect 3, wherein the method comprises administering to the patient a therapeutically effective amount of one or more lamining-5-binding integrins selected from the group consisting of alpha3beta1, alpha6beta1, and alpha6beta4.

5. The method of any one of aspects 1-4, wherein the method comprises administering a therapeutically effective amount of an antibody that binds laminin-5 to the patient.

6. The method of aspect 5, wherein the laminin-5 antibody detectably binds the γ2 chain of laminin-5.

7. The method of aspect 5, wherein the laminin-5 antibody detectably binds the α3 chain of laminin-5.

8. The method of aspect 5, wherein the laminin-5 antibody detectably binds the β3 chain of laminin-5.

9. The method of any of aspects 1-8, wherein the method comprises administering a therapeutically effective amount of an antibody that binds a laminin-5-binding integrin to the patient.

10. The method of aspect 9, wherein the antibody binds an integrin selected from the group consisting of alpha3beta1, alpha6beta1, and alpha6beta4.

11. The method of any of the preceding aspects, wherein the patient suffers from ulcerative colitis.

12. A method of reducing the risk of developing cancer in a patient suffering from an inflammatory bowel disease comprising administering to the patient a composition comprising a laminin-5-associated antibody, a laminin-5 binding protein, or both in an amount sufficient to detectably reduce the risk of developing cancer in a population of similar patients.

13. A method of inducing, promoting, and/or enhancing a physiological response associated with the prevention and/or treatment of cancer in a patient suffering from an inflammatory bowel disease and identified as having or at risk of developing a inflammatory bowel disease-associated cancer comprising administering to the patient a composition comprising a laminin-5-associated antibody, a laminin-5 binding protein, or both in an amount sufficient to detectably induce the physiological response.

14. The method of aspect 12 or aspect 13, wherein the method comprises administering a therapeutically effective amount of a laminin-5-binding integrin to the patient.

15. The method of aspect 14, wherein the method comprises administering to the patient a therapeutically effective amount of one or more lamining-5-binding integrins selected from the group consisting of alpha3beta1, alpha6beta1, and alpha6beta4.

15. The method of any one of aspects 12-14, wherein the method comprises administering a therapeutically effective amount of an antibody that binds laminin-5 to the patient.

16. The method of aspect 15, wherein the laminin-5 antibody detectably binds the γ2 chain of laminin-5.

17. The method of aspect 15, wherein the laminin-5 antibody detectably binds the α3 chain of laminin-5.

18. The method of aspect 15, wherein the laminin-5 antibody detectably binds the β3 chain of laminin-5.

19. The method of any of aspects 12-18, wherein the method comprises administering a therapeutically effective amount of an antibody that binds a laminin-5-binding integrin to the patient.

20. The method of aspect 19, wherein the antibody binds an integrin selected from the group consisting of alpha3beta1, alpha6beta1, and alpha6beta4.

21. The method of any of aspects 12-20, wherein the patient suffers from ulcerative colitis.

22. The method of any of aspects 12-21, wherein the patient has been identified as suffering from a cancer and the method comprises administering an anti-cancer agent or the patient has been identified as being at substantial risk of developing an inflammatory bowel disease-associated cancer and the method comprises administering a cancer preventative agent to the patient.

23. A method of reducing the risk of developing, preventing the development, or reducing the spread, growth, or spread and growth of renal cysts in a patient identified as having such a cyst or having a substantial risk of developing such a cyst comprising administering a composition comprising a laminin-5-associated antibody, a laminin-5 binding protein, or both to the patient in an amount sufficient to detectably reduce the risk of developing, preventing the development, or reducing the spread, growth, or spread and growth of renal cysts in a population of similar patients.

24. A method of inducing, promoting, and/or enhancing a physiological response in a patient identified as having a renal cyst or having a substantial risk of developing a renal cyst comprising administering to a composition comprising a laminin-5-associated antibody, a laminin-5 binding protein, or both to the patient in an amount sufficient to detectably induce, promote, and/or enhance the physiological response.

25. A method of ameliorating a disease state associated with the growth and/or spread of renal cysts in a patient having such cysts comprising administering a composition comprising a laminin-5-associated antibody, a laminin-5 binding protein, or both to the patient in an amount sufficient to detectably ameliorate the disease state.

26. The method of any one of aspects 23-25, wherein the method comprises administering a therapeutically effective amount of a laminin-5-binding integrin to the patient.

27. The method of aspect 26, wherein the method comprises administering to the patient a therapeutically effective amount of one or more lamining-5-binding integrins selected from the group consisting of alpha3beta1, alpha6beta1, and alpha6beta4.

28. The method of any one of aspects 23-27, wherein the method comprises administering a therapeutically effective amount of an antibody that binds laminin-5 to the patient.

29. The method of aspect 28, wherein the laminin-5 antibody detectably binds the γ2 chain of laminin-5.

30. The method of aspect 28, wherein the laminin-5 antibody detectably binds the α3 chain of laminin-5.

31. The method of aspect 28, wherein the laminin-5 antibody detectably binds the β3 chain of laminin-5.

32. The method of any of aspects 23-31, wherein the method comprises administering a therapeutically effective amount of an antibody that binds a laminin-5-binding integrin to the patient.

33. The method of aspect 32, wherein the antibody binds an integrin selected from the group consisting of alpha3beta1, alpha6beta1, and alpha6beta4.

34. The method of any of aspects 23-33, wherein the method comprises administering at least one secondary anti-renal cyst agent to the patient.

35. A method of reducing the risk of developing, delaying the onset of, reducing the severity, preventing and/or treating irritable bowel syndrome in a patient suffering from irritable bowel syndrome or identified as being of substantial risk of developing irritable bowel syndrome comprising administering to the host a therapeutically or prophylactically effective amount of a laminin-5-associated antibody, a laminin-5 binding protein, or both to achieve the desired outcome(s).

36. A method of promoting, enhancing, and/or inducing one or more physiological effects in a patient associated with the prevention and/or treatment of irritable bowel syndrome comprising administering to the patient a composition comprising an amount of a laminin-5-associated antibody, a laminin-5 binding protein, or both in an amount sufficient to detectably promote, enhance, and/or induce the physiological effect(s).

37. The method of any one of aspects 35-36, wherein the method comprises administering a therapeutically effective amount of a laminin-5-binding integrin to the patient.

38. The method of aspect 37, wherein the method comprises administering to the patient a therapeutically effective amount of one or more lamining-5-binding integrins selected from the group consisting of alpha3beta1, alpha6beta1, and alpha6beta4.

39. The method of any one of aspects 35-38, wherein the method comprises administering a therapeutically effective amount of an antibody that binds laminin-5 to the patient.

40. The method of aspect 39, wherein the laminin-5 antibody detectably binds the γ2 chain of laminin-5.

41. The method of aspect 39, wherein the laminin-5 antibody detectably binds the α3 chain of laminin-5.

42. The method of aspect 39, wherein the laminin-5 antibody detectably binds the β3 chain of laminin-5.

43. The method of any of aspects 35-42, wherein the method comprises administering a therapeutically effective amount of an antibody that binds a laminin-5-binding integrin to the patient.

44. The method of aspect 43, wherein the antibody binds an integrin selected from the group consisting of alpha3beta1, alpha6beta1, and alpha6beta4.

45. The method of any one of aspects 35-44, wherein the method comprises administering at least one secondary anti-irritable bowel syndrome agent to the patient.

46. A method of reducing cancer progression in a mammalian host afflicted with cancer comprising administering a composition comprising a synergistically effective combination of a laminin-5-associated antibody, a laminin-5 binding protein, or both and one or more anti-cancer agents to the host in an amount sufficient to detectably reduce the progression of the cancer.

47. A method of increasing the ratio of quiescent to invasive neoplastic cells in a mammalian host comprising administering a therapeutically effective amount of a composition comprising a synergistically effective combination of a laminin-5-associated antibody, a laminin-5 binding protein, or both and one or more anti-cancer agents to the host so as to increase the ratio of quiescent to invasive cells in the host.

48. A method of preventing the formation of cancer associated tubular networks in a mammalian host comprising administering a therapeutically effective amount of a composition comprising a synergistically effective combination of a laminin-5-associated antibody, a laminin-5 binding protein, or both and one or more anti-cancer agents to the host so as to detectably reduce the risk of developing cancer associated tubular networks, prolong the onset of cancer associated tubular networks, and/or reduce the number of expected cancer associated tubular networks formed in the host.

49. A method of reducing the invasive potential of cancer cells in a mammalian host comprising administering a therapeutically effective amount of a composition comprising a synergistically effective combination of a laminin-5-associated antibody, a laminin-5 binding protein, or both and one or more anti-cancer agents to the host so as to detectably reduce the invasive potential of the cancer cells.

50. A method of reducing cell migration, reducing tumor growth, reducing neoplastic or pre-neoplastic cell division, or any combination thereof in a mammalian host comprising administering a therapeutically effective amount of a composition comprising a synergistically effective combination of a laminin-5-associated antibody, a laminin-5 binding protein, or both and one or more anti-cancer agents to the host so as to achieve the desired outcome(s).

51. A method for modulating MAP kinase activity in neoplastic or preneoplastic cells of a mammalian host comprising contacting the cells with a physiologically effective amount of a composition comprising a synergistically effective combination of a laminin-5-associated antibody, a laminin-5 binding protein, or both and one or more anti-cancer agents to the cells so as to detectably modulate MAP kinase activity in the cells.

52. A method for reducing the risk of developing a cancer, reducing the time to onset of a cancerous condition, reducing the severity of a cancer diagnosed in the early stages, and/or reducing the affected area of a cancer upon development thereof in a mammalian host, comprising administering to a host a prophylactically effective amount of a composition comprising a synergistically effective combination of a laminin-5-associated antibody, a laminin-5 binding protein, or both, and one or more anti-cancer prophylactic agents so as to achieve the desired physiological effect(s).

53. The method of any one of aspects 46-52, wherein the method comprises administering a therapeutically effective amount of a laminin-5-binding integrin to the patient.

54. The method of aspect 54, wherein the method comprises administering to the patient a therapeutically effective amount of one or more lamining-5-binding integrins selected from the group consisting of alpha3beta1, alpha6beta1, and alpha6beta4.

55. The method of any one of aspects 46-54, wherein the method comprises administering a therapeutically effective amount of an antibody that binds laminin-5 to the patient.

55. The method of aspect 55, wherein the laminin-5 antibody detectably binds the γ2 chain of laminin-5.

56. The method of aspect 55, wherein the laminin-5 antibody detectably binds the α3 chain of laminin-5.

57. The method of aspect 55, wherein the laminin-5 antibody detectably binds the β3 chain of laminin-5.

58. The method of any of aspects 46-57, wherein the method comprises administering a therapeutically effective amount of an antibody that binds a laminin-5-binding integrin to the patient.

59. The method of aspect 58, wherein the antibody binds an integrin selected from the group consisting of alpha3beta1, alpha6beta1, and alpha6beta4.

60. A method of promoting the sale and/or use of a compound according to any of the preceding aspects, or otherwise described herein, comprising distributing information related to the use of the compound in the prevention or treatment of any condition or combination of conditions recited in any of the foregoing aspects or described elsewhere herein.

61. A pharmaceutical product comprising (a) a composition according to any of the foregoing aspects or elsewhere described herein, (b) a pharmaceutically acceptable carrier, vehicle, excipient, diluent, preservative, stabilizer, binder, flavoring agent, an antioxidant, a colorant, adjuvant, disintegrating agent, solvent, a solubilizer, a suspending agent, a isotonizing/isotonic agent, a buffer, a soothing agent, or combination of any thereof (including any multiples thereof—e.g., two diluents), and, optionally, (c) a notice associated with said container in form prescribed by a governmental agency regulating the manufacture, use, or sale of pharmaceuticals, which notice is reflective of approval by said agency of said pharmaceutical product for human or veterinary administration to treat at least one condition recited in any of the foregoing aspects or other condition or disease described herein.

62. A method of screening candidate compositions for biological activity associated with the prevention and/or treatment of inflammatory bowel disease in a mammal comprising:

(a) providing a composition comprising a laminin-5-associated antibody, a laminin-5 binding protein, or both, in an amount expected to result in a response in a model of inflammatory bowel disease that is predictive of a correlated therapeutic or prophylactic effect in a mammal,

(b) subjecting the composition to analysis by the model, and

(c) assessing the therapeutic and/or prophylactic potential the composition by assessing the effect of the composition in the model.

63. A method of screening candidate compositions for biological activity associated with reducing the spread and/or growth of renal cysts in a mammal comprising:

(a) providing a composition comprising a laminin-5-associated antibody, a laminin-5 binding protein, or both, in an amount expected to result in a response in a model of Autosomal Dominant Polycystic Kidney Disease that is predictive of a correlated therapeutic or prophylactic effect in a mammal,

(b) subjecting the composition to analysis by the model, and

(c) assessing the therapeutic and/or prophylactic potential the composition by assessing the effect of the composition in the model.

64. A method of screening candidate compositions for biological activity associated with treating or preventing irritable bowel syndrome comprising:

(a) providing a composition comprising a laminin-5-associated antibody, a laminin-5 binding protein, or both, in an amount expected to result in a response in a model of irritable bowel syndrome that is predictive of a correlated therapeutic or prophylactic effect in a mammal,

(b) subjecting the composition to analysis by the model, and

(c) assessing the therapeutic and/or prophylactic potential the composition by assessing the effect of the composition in the model.

65. A method of screening candidate synergistic combinations of anti-cancer agents comprising:

(a) providing a composition comprising a combination of a laminin-5-associated antibody, a laminin-5 binding protein, or both, and at least one secondary anti-cancer agent in amounts expected to result in a response in a model of that is predictive of a correlated therapeutic or prophylactic effect in a mammalian host,

(b) obtaining a predicted cumulative effect for the combination,

(b) subjecting the composition to analysis by the model, and

(c) assessing whether the combination results in a result that is substantially greater than the predicted cumulative effect for the combination so as to identify a synergistic combination of anti-cancer agents.

66. The method of aspect 65, wherein the secondary anti-cancer agent is selected from the agents recited herein.

67. The method of any of the preceding aspects, wherein the method is performed using one or more non-Ln-5-associated antibody, non-Ln-5 binding protein, Ln-5 modulators.

68. The method of aspect 67, wherein the Ln-5 modulator is a siRNA, an antisense molecule, or a small molecule Ln-5 modulator.

69. A method of treating inflammatory bowel disease comprising the step of administering to a subject suffering from inflammatory bowel disease a composition comprising an effective amount of a laminin-5 modulator.

70. A method of reducing the risk of inflammatory bowel disease comprising the step of administering to a subject at risk for inflammatory bowel disease a composition comprising an effective amount of a laminin-5 modulator.

71. A method of reducing the risk of inflammatory bowel disease-associated colorectal carcinoma comprising the step of administering to a subject suffering from inflammatory bowel disease a composition comprising an effective amount of a laminin-5 modulator.

72. The method of any of aspects 69 to 71, wherein the inflammatory bowel disease is ulcerative colitis.

73. The method of any of aspects 69 to 71, wherein the inflammatory bowel disease is Crohn's disease.

74. The method of any of aspects 69-73, comprising the administration of at least one additional anti-inflammatory bowel disease agent.

75. A method of treating irritable bowel syndrome comprising the step of administering to a subject suffering from irritable bowel syndrome a composition comprising an effective amount of a laminin-5 modulator.

76. A method of reducing the risk of irritable bowel syndrome comprising the step of administering to a subject at risk for irritable bowel syndrome a composition comprising an effective amount of a laminin-5 modulator.

77. The method of any of aspects 75 and 76, comprising the administration of at least one additional anti-irritable bowel syndrome agent.

78. A method of treating polycystic kidney disease comprising the step of administering to a subject identified as suffering from polycystic kidney disease a composition comprising an effective amount of a laminin-5 modulator.

79. A method of reducing the progression of polycystic kidney disease comprising the step of administering to a subject identified as suffering from autosomal dominant polycystic kidney disease a composition comprising an effective amount of a laminin-5 modulator.

80. The method of any of aspects 78 and 79, comprising the administration of at least one additional anti-renal cyst agent.

81. A method of treating cancer comprising the step of administering to a subject suffering from cancer a composition comprising a synergistically effective combination of a laminin-5 modulator and at least one secondary anti-cancer agent.

82. A method of reducing tumor growth comprising the step of administering to a subject suffering from a tumor a composition comprising a synergistically effective combination of a laminin-5 modulator and at least one secondary anti-cancer agent.

83. A method of reducing tumor invasiveness or metastatic growth comprising the step of administering to a subject suffering from a primary tumor a composition comprising a synergistically effective combination of a laminin-5 modulator and at least one secondary anti-cancer agent.

84. The method of any of aspects 81-83, wherein the secondary anti-cancer agent is selected from the group consisting of Melphalan, Busulfan, Cis-platin, Carboplatin, Cyclophosphamide, Ifosphamide, Dacarbazine, Chlorambucil, Thiotepa, Lomustine, Temozolamide, Cytarabine, Azathioprine, Fludarabine phosphate, Fludarabine, 5-fluorouracil, Gemcitabine, Hydroxyurea, Cladribine, Thioguanine, Methotrexate, Capecitabine, Vinorelbine, Vincristine, Vinblastine, Docetaxel, Paclitaxel, Doxorubicin, Amsacrine, Irinotecan, Daunorubicin, Epirubicin, Mitomycin, Mitoxantrone, Idarubicin, Teniposide, Etoposide, Topotecan, Actinomycin, Bleomycin, aspariginase, Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin, Goserelin, and any other cancer agent mentioned herein, as well as combinations thereof.

85. The method of any of aspects 69-84, wherein the laminin-5 modulator is selected from the group consisting of an antibody binding laminin-5, a laminin-5 binding protein, an siRNA, an antisense molecule, or a small molecule Ln-5 modulator.

86. The method of aspect 85, wherein the laminin-5 modulator is an antibody binding the laminin-5 gamma2 chain.

87. The method of aspect 85, wherein the laminin-5 modulator is an siRNA directed against a nucleic acid encoding the laminin-5 gamma2 chain.

88. The use of a laminin-5 modulator in the preparation of a medicament for treating inflammatory bowel disease.

89. The use of a laminin-5 modulator in the preparation of a medicament for treating inflammatory bowel disease-associated colorectal carcinoma.

90. The use of a laminin-5 modulator in the preparation of a medicament for treating irritable bowel syndrome.

91. The use of effective amounts of a laminin-5 modulator and at least one secondary anti-cancer agent in the preparation of a medicament for treating cancer.

92. The use of effective amounts of a laminin-5 modulator and at least one secondary anti-cancer agent in the preparation of a medicament for reducing tumor growth.

93. The use of effective amounts of a laminin-5 modulator and at least one secondary anti-cancer agent in the preparation of a medicament for reducing the risk for metastatic growth of a primary tumor.

94. The use of any of aspects 88-93, wherein the laminin-5 modulator is selected from the group consisting of an antibody directed against laminin-5, a laminin-5 binding protein, an siRNA, an antisense molecule, or a small molecule Ln-5 modulator.

95. The use of aspect 94, wherein the laminin-5 modulator is an antibody directed against the laminin-5 gamma2 chain.

96. The use of aspect 94, wherein the laminin-5 modulator is an siRNA directed against a nucleic acid encoding the laminin-5 gamma2 chain.

97. A treatment for inflammatory bowel disease, polycystic kidney disease, or irritable bowel syndrome, said inflammatory bowel disease including ulcerative colitis and Crohn's disease, comprising means for modulating Ln-5.

98. A method of promoting the sale of an Ln-5 modulator comprising providing information relating to the use of Ln-5 in the treatment of inflammatory bowel disease, polycystic kidney disease, or irritable bowel syndrome, said inflammatory bowel disease including ulcerative colitis and Crohn's disease, to health care professionals and/or organizations including, but not limited to, prescribing physicians, formulary managers, insurance officials, research scientists, and/or patients.

These aspects are more fully described in, and additional aspects, features, and advantages of the invention will be apparent from, the description of the invention provided herein.

### BRIEF DESCRIPTION OF THE DRAWINGS

FIG. 1. Paraffin sections of colon from (A) IBD and (B) normal control mice were immunostained for Ln-5 γ2 chain expression. Arrowheads indicate Ln-5 γ2 immunopositive epithelial cells.

FIG. 2. Paraffin sections of IBD colon were immunostained for (A) Ln-5 γ2 chain and (B) BrdU. Arrows indicate Ln-5 γ2-immunopositive, BrdU-negative epithelial cells.

FIG. 3. Western blot analysis of the effect of siRNA on Ln-5 γ2 expression in an in vitro model.

FIG. 4. Cell death of SW480 cells transfected with anti-LN5 gamma2 siRNA and treated with chemotherapeutic compounds.

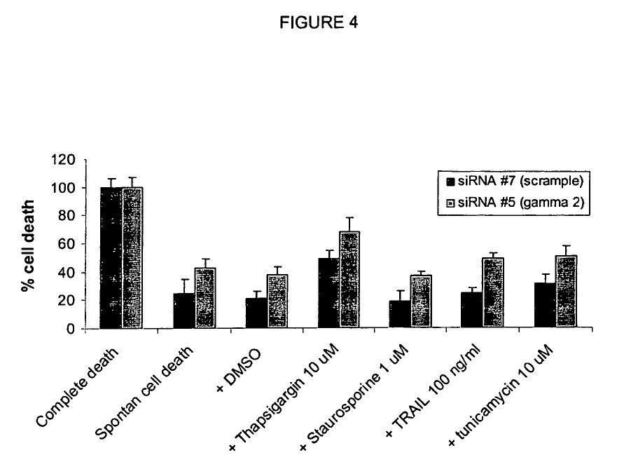

### DESCRIPTION OF THE INVENTION

The invention described herein is based, in part, on the discovery that modulators of laminin-5 (Ln-5) can be used to promote, enhance, and/or induce physiological responses associated with the treatment and/or prevention of various diseases and disorders including, for example, IBD, IBD-associated cancers, irritable bowel syndrome (“IBS”), and PKD, caused by, e.g., autosomal polycystic kidney disease (ADPKD). In one embodiment, modulators of laminin-5 are used to promote, enhance, and/or induce physiological responses associated with the treatment and/or prevention of diseases and disorders that, heretofore, were not known to be associated with laminin-5 modulation. Methods of promoting the treatment and/or prevention of these and other diseases and disorders by administering a Ln-5 modulator under conditions such that one or more functions of Ln-5 and/or one or more functions of one or more biological molecules associated with Ln-5 (normally and/or in the relevant disease state(s) in the applicable patient or host) are at least partially (e.g., at least substantially, or essentially entirely) abrogated are another important feature of this invention. For example, the administration of an Ln-5 modulator to an IBD patient at risk for developing colon cancer can lower the cancer risk and/or reverse the IBD disease state to a milder condition. Additionally, an Ln-5 modulator can slow down or inhibit the progression of ADPKD, and combination treatments based on an Ln-5 modulator and a second anti-cancer agent can be used to treat cancer.

The invention described herein also provides novel compositions comprising one or more Ln-5 modulators that can be used in such methods as well as in the prevention and/or treatment of cancer, generally (related methods also are provided). Additional important general features of the invention include, but are not limited to, methods of identifying Ln-5 modulators, preparing such compositions, and promoting the sale of Ln-5 modulator compositions in association with particular therapeutic, prophylactic and/or diagnostic uses.

As described herein, Ln-5 modulation can, for example, be achieved using Ln-5 modulators such as antibodies against Ln-5 and anti-sense technology. Ln-5 antibodies are described in, e.g., U.S. Pat. Nos. 5,660,982 and 6,143,505, U.S. Patent Application Nos. 2004/0120959, 2003/0100529 and 2002/0052307, and European Patent 0 784 703, and Ln-5 antisense nucleic acids and other relevant molecules and methods are described in, e.g., U.S. Patent Application Publication No. 20030224993 and Seftor et al., Cancer Res. 2001 Sep. 1; 61 (17):6322-7.

Laminin-5 (“Ln-5”) is a basement membrane extracellular matrix macromolecule that provides an attachment substrate for both adhesion and migration in a wide variety of cell types. Structurally, Ln-5, like other laminins, is composed of alpha, beta, and gamma chain subunits (Ln-5, specifically, has the general structure α3β3γ2). Ln-5 is mainly expressed in epithelial basement membranes, where its biological functions involve anchorage and locomotion of cells. Expression of the Ln-5 γ2 chain has also been found at the invasive front of tumors such as colorectal cancers, as well as in UC and ADPKD tissue samples (Habermann et al., Scand J Gastroenterol 2001; 36:751-8 and Joly et al., Am J Pathol 2003; 163:1791-1800).

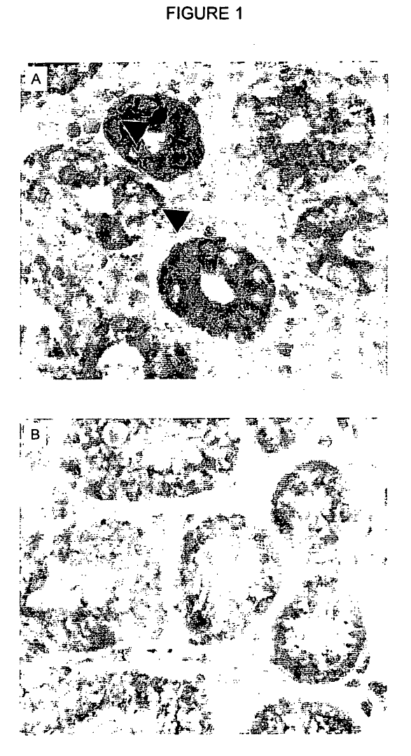

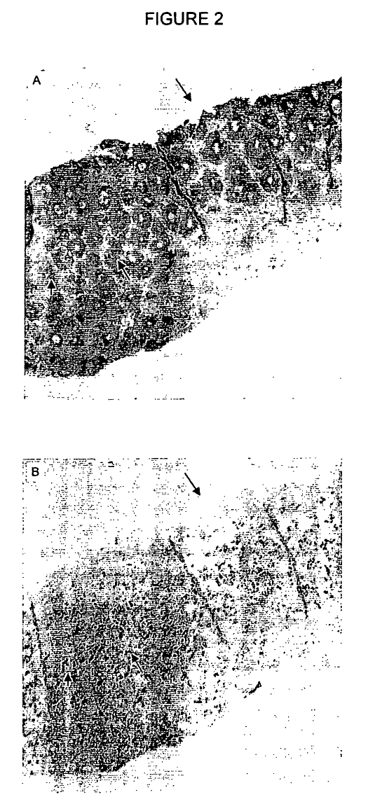

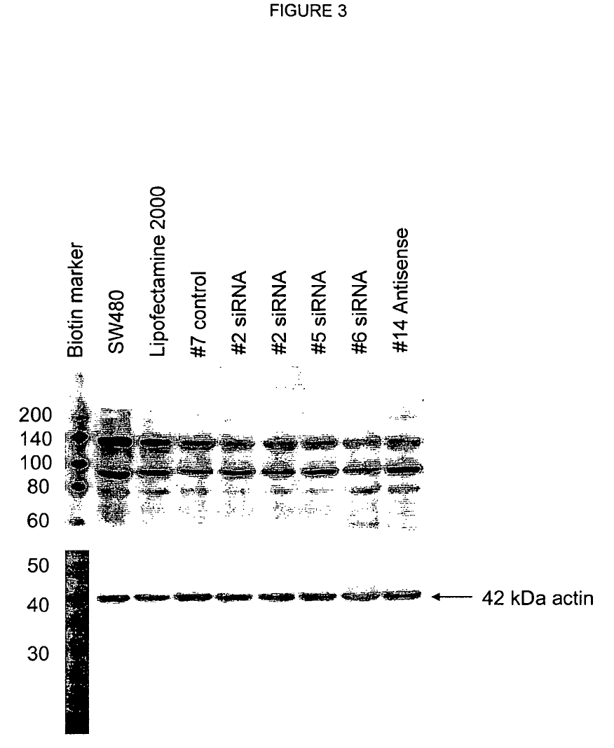

As shown in Example 1 and FIGS. 1 and 2, in an animal model of IBD, Ln-5 γ2-expression in IBD inflicted tissue was observed in crypt cells and parts of the crypt. Example 2 reports that in vitro Ln-5 modulation based on siRNA administration successfully reduced the expression of the Ln-5 γ2 chain, as shown in FIG. 3. Example 3 shows that the use of siRNA to Ln-5 γ2 chain increases spontaneous cell death and the increased cell death is retained also when siRNA is administered in combination with other anti-cancer compounds.

Ln-5 Modulators

In general, the methods of the invention can be practiced using any suitable agent that modulates the biological activity of Ln-5 (“Ln-5 modulator”) in a manner suitable for achieving the desired physiological result(s). In a particular embodiment, a combination of two or more different Ln-5 modulators are combined to achieve the desired physiological result(s).

As described herein, “Ln-5 modulation” includes, but is not limited to, reduction of Ln-5 expression, reduction of the binding of Ln-5 and an Ln-5 ligand, and/or reduction of the in vivo or in vitro growth potential or migratory or invasive capacity of a cell expressing Ln-5, or any combination of the foregoing. Ln-5 ligands and Ln-5 binding proteins, that may serve as Ln-5 modulators, are exemplified below.

Ln-5 modulators include, for example, anti-Ln-5 antibodies (“Ln-5 antibodies”) and antibodies that bind to other peptides, proteins, and/or polypeptides that interact with Ln-5 (in the normal and/or disease state in a target host). Such non-immunoglobulin Ln-5-associated polypeptides, peptides, and proteins can be referred to as “Ln-5 binding proteins” (the terms polypeptide, peptide, and protein are used interchangeably throughout unless otherwise stated or clearly contradicted by context and include derivatives of such polypeptides, such as polypeptides modified by glycosylation, acylation, and the like as described in, e.g., International Patent Applications WO 02/083851, WO 03/048185, and WO 03/102166 and relevant references cited therein). Exemplary anti-LN-5 antibodies include 5D5 and 6C12, described in International Patent Application WO2004/039401 and U.S. Patent Application 2004/0120959, hereby incorporated by reference in their entireties. Collectively, such anti-Ln-5 antibodies and antibodies against Ln-5 binding proteins can be referred to as Ln-5-associated antibodies.

Additional Ln-5 modulators include functional nucleic acid molecules such as antisense nucleic acids (typically oligonucleotides) that are targeted, for example, against polypeptides that modulate Ln-5 activity in vivo (e.g., Ln-5 itself as well as polypeptides that modify Ln-5 in association with cancer progression, development of IBD, development of ADPKD, and the like—examples of which may include certain integrins, MMP-2, and/or BMP-1 as described further herein); protein and/or nucleic acid aptamers (including intramers—see, e.g., Famulok et al., Chem Biol. 2001 October; 8(10):931-9), interfering RNA molecules (e.g., small or short interfering RNA (“siRNA”) molecules, which are typically double-stranded (ds), and/or larger dsRNA molecules designed for RISC cleavage into relevant siRNAs); ribozymes, triplex-forming molecules, external guide sequences, and other “gene silencing agents” known in the art. The functional nucleic acid molecules can act as affectors, inhibitors, modulators, and stimulators of a specific activity possessed by a target molecule, so that Ln-5 activity is modulated, or the functional nucleic acid molecules can possess a de novo activity independent of any other molecules.

In other aspects, an Ln-5 modulator can be an agent that upregulates production of particular types of Ln-5 molecules and/or Ln-5 binding proteins, such as a demethylating agent, see, e.g., Urburadka et al., Clinical Cancer Research Vol. 9, 2665-2672, July 2003, a gene activation agent (e.g., a promoter and/or enhancer of Ln-5 or Ln-5-associated protein expression that is integrated into the genome of target cells by homologous recombination techniques), and the like. A Ln-5 modulator also can be a Ln-5 modulating “small molecule” compound (a small molecule is generally considered an organic compound having a molecular weight of less than about 1,000 Daltons and more typically less than about 500 Daltons—with typical exclusion of nucleic acids and peptides (particularly of any significant length) but inclusion of sugars, individual amino acids, lipids, vitamins, and non-peptide hormones), such as, for example, a Ln-5 binding heparin. Typically, a small molecule Ln-5 modulator will bind to and disrupt interaction between Ln-5 and an Ln-5 binding protein under physiological conditions associated with the relevant disease or pre-disease state in the subject.

An Ln-5-associated antibody refers to an immunoglobulin molecule or fragment or derivative thereof that has the ability to specifically bind Ln-5, a component thereof (e.g., the α3, β3, or γ2 chain or a domain thereof—such as domain III of the γ2 chain), or an Ln-5 binding protein. Unless otherwise stated or clearly contradicted by context, included within this definition of antibody are antibody fragments that retain the ability to specifically bind to Ln-5 or a portion thereof. Examples of binding fragments encompassed within the term “antibody” include, but are not limited to, the following: (i) a Fab fragment, a monovalent fragment consisting of the VL, VH, CL, and CH I domains; (ii) F(ab)₂ and F(ab′)₂ fragments, a bivalent fragment comprising two Fab fragments linked by a disulfide bridge at the hinge region; (iii) a Fd fragment consisting of the VH and CH1 domains; (iv) a dsFv fragment, obtained by binding polypeptides in which one amino acid residue of each of VH and VL is substituted with a cysteine residue via a disulfide bond between the cysteine residues (see Reiter et al. (Protein Engineering, 7, 697 (1994)); (v) a dAb fragment (see, e.g., Ward et al., (1989) Nature 341:544-546), which consists of a VH domain; (vi) an isolated complementarity determining region (CDR), and (viii) linear antibodies, which comprise a pair of tandem Fd segments that form a pair of antigen binding regions (see, e.g., Zapata et al. Protein Eng. 8(10):1057-1062 (1995)). Furthermore, although the two domains of the Fv fragment, VL and VH, typically are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antibody”. Other forms of single chain antibodies, such as diabodies are also encompassed by the term “antibody” unless otherwise stated. Diabodies are bivalent and bispecific antibodies in which VH and VL domains are expressed on a single polypeptide chain, but are separated by a linker that is too short to allow for pairing between the two domains on the same chain, thereby forcing the domains to pair with complementary domains of another chain so as to create two antigen binding sites (see e.g., Holliger, P., et al. (1993) Proc. Natl. Acad. Sci. USA 90:6444-6448; Poljak, R. J., et al. (1994) Structure 2:1121-1123, EP 404,097 and WO 93/11161).

Also contemplated are “fully human antibodies”, which are antibodies having both variable and constant regions derived from human germ line immunoglobulin sequences. Such antibodies can be generated in humanized transgenic animals comprising human immunoglobulin loci and native immunoglobulin gene deletions, such as in a XenoMouse™ (Abgenix—Fremont, Calif., USA) (see, e.g., Green et al. Nature Genetics 7:13-21 (1994); Mendez et al. Nature Genetics 15:146-156 (1997); Green and Jakobovits J. Exp. Med. 188:483-495 (1998); European Patent No., EP 0 463 151 B1; International Patent Application Nos. WO 94/02602, WO 96/34096; WO 98/24893, WO 99/45031, WO 99/53049, and WO 00/037504; and U.S. Pat. Nos. 5,916,771, 5,939,598, 5,985,615, 5,998,209, 5,994,619, 6,075,181, 6,091,001, 6,114,598 and 6,130,364) or transgenic vertebrates comprising a minilocus of human Ig-encoding genes.

In certain aspects, the Ln-5-associated antibody used in the various methods of the invention is a “humanized antibody.” Humanized antibodies are recombinant antibodies in which complementarity determining region (CDR) sequences or variants thereof derived from the germ line of another mammalian species, such as a mouse, have been grafted into human “framework” sequences. In one aspect of the invention, the isolated humanized monoclonal antibody comprises CDR amino acid sequences derived from a monoclonal antibody. In a further aspect of the invention, the isolated humanized monoclonal antibody has CDR amino acid sequences derived from a mouse monoclonal antibody.

In certain aspects, the Ln-5-associated antibody used in the various methods of the invention is a chimeric antibody, by which is meant an antibody wherein specific parts of the molecule have been changed so that the molecule has reduced immunogenicity in humans. Such antibodies and methods of making them are well known in the art.

The antibodies used in the various methods of the invention can be of natural or synthetic origin. Natural antibodies can be monoclonal or polyclonal, both of which are well known in the art. In connection with synthetic antibodies, antibodies can be non-naturally occurring immunoglobulin molecules that vary by one or more amino acid substitutions, replacements, additions, and/or deletions, provided that such immunoglobulin molecules retain their ability to bind Ln-5, an Ln-5 binding protein, or portions of either thereof. Synthetic antibodies also can be “derivatives” of Ln-5-associated antibodies, varying by the addition of modified amino acids, chemical moieties, etc. Unless otherwise stated, the various methods of the invention involving an Ln-5-associated antibody can be practiced with combinations of Ln-5-associated antibodies of different chemical composition, antigen specificity, or both.

Typically, the Ln-5-associated antibody used in the methods of the invention is an antibody against Ln-5. Any suitable Ln-5 antibody can be used in such methods. Thus, for example, the methods of the invention may be practiced with an Ln-5 antibody detectably binds the γ2 chain of Ln-5, an Ln-5 antibody detectably binds the α3 chain of Ln-5, or an Ln-5 antibody detectably binds the β3 chain of Ln-5.

An Ln-5 binding protein refers to a non-immunoglobulin polypeptide that detectably binds Ln-5 under normal and/or disease state physiological conditions. Examples of Ln-5 binding proteins include type VII collagen, fibulin-1, fibulin-2, BP180, syndecan-4, dystroglycan, nidogen-1, matrix metalloproteinases (e.g., MMP-1, MMP-2, MMP-9, MMP-10 (Stromelysin-2) and MMP-14 (also known as MT1-MMP)), tissue inhibitor of matrix metalloproteinase-1 (TIMP-1) and TIMP-2, laminin-6, laminin-7, EGF-R, Rho GTPases, phosphorylated hsp-27, p300, a cytokeratin, E-cadherin, the 67 kDa laminin receptor, and bone morphogenic protein 1 (BMP-1). In one aspect, the Ln-5 binding protein is an Ln-5-binding integrin. In such aspects, the Ln-5 binding protein can be, for example, selected from the group consisting of the alpha3beta1 (α3β1), alpha6beta1 (α6β1), alpha2beta1 (α2β1) and alpha6beta4 (α6β4) integrins.

An Ln-5 binding protein also can be a fragment of such proteins that retains Ln-5 binding capabilities, or a synthetic variant (homolog) of such proteins that retains the ability to bind Ln-5, or a derivative of such proteins or variants. Retention of Ln-5 binding ability can be detected in an ELISA assay by immobilizing the putative Ln-5 binding protein to the surface and after washing adding soluble Ln-5 at graded concentrations (e.g., 0, 1 ng, 10 ng, 100 ng, 1 μg, 10 μg) incubate at room temperature (20 C) for 1 hour to 24 hours before washing with a standard ELISA wash buffer and subsequent detection of laminin-5 by using an anti-Ln-5 antibody either coupled to a fluorophore or by itself, followed by the addition of a secondary anti mouse antibody with suitable detection like horse radish peroxidase washing to ensure specificity and finally detection. Binding between Ln-5 and a putative Ln-5 binding protein can also be detected by including membrane bound protein or proteins (such as, e.g., an integrin heterodimer) in a liposome, loading the liposome interior with radioactivity and then detect the radioactivity bound to a Ln-5 coated surface (10 nM) or laminin 10/11 (10 nM) to discriminate for specific binding. Such methods are described in more detail in Nishiuchi et al. (J. Biochem. 2003; 134:497-504). Binding of Ln-5 and a putative Ln-5 binding protein can also be detected by using FACS analysis of a cell line expressing Ln-5 and/or the Ln-5 binding protein where either the Ln-5 binding protein or Ln-5 itself is labelled by FITC or another fluorophore to detect cells being stained upon the binding interaction. Unspecific signal can be determined using a control cell staining where the expression of Ln-5 or the Ln-5 binding protein is lacking (the unstained protein needs to be missing). Disruption of binding of Ln-5 and a Ln-5 binding protein can be determined by a reduced or lack of specific binding interaction as determined by methods in the art exemplified but not limited to the list mentioned above. As defined herein, a putative Ln-5 binding protein which has a higher detection value in such an assay than a suitable control (e.g., unspecific binding test described above, or using the same assay but either excluding Ln-5 or using an irrelevant control protein instead of Ln-5) is an Ln-5 binding protein. Unless otherwise stated, it may be permissible to use combinations of such Ln-5 binding proteins in the practice of the methods of the invention. An example could be the combination of extracellular domain of an alpha and a beta integrin subunit in such a binding assay.

In other aspects, the Ln-5-associated antibody is an antibody against an Ln-5 binding protein. Thus, for example, the Ln-5-associated antibody can be any antibody against an Ln-5-binding integrin such as alpha2beta1, alpha3beta1, alpha6beta1, or alpha6beta4 integrin, including antibodies binding to the beta1 domain of the beta-subunit or antibodies binding to the alpha1 domain/beta propeller in the alpha subunit of an integrin. See also Xiao et al., Nature (2004) vol 432 page 59-67. In other aspects, the Ln-5-associated antibody may be an antibody against MMP-2 or an antibody against BMP-1.

Peptide Ln-5 modulators can be administered directly or can be expressed in the host by any suitable genetic expression system. Thus, for example, the various methods of the invention can be practiced by the delivery of a nucleic acid that, upon expression, results in the production of a Ln-5 binding protein (e.g., a wild-type Ln-5 binding protein or a Ln-5 binding variant of such a protein, and/or a fragment of either thereof or fusion protein comprising any thereof) and/or a Ln-5-associated antibody. Such nucleic acids can be advantageously delivered by way of a suitable vector, such as a linear expression element, plasmid, cosmid, or viral vector such as an adenoviral vector (e.g., a wild-type or modified adenovirus—such as a replication-deficient and targeted adenovirus), pox viral vector, herpes viral vector, lentiviral vector, adeno-associated virus (AAV), and the like. As already mentioned, expression of such peptides also can be accomplished by the administration of agents that upregulate such expression, either in terms of transduction factors for such agents, gene activation, or other suitable technique.

Antisense Ln-5 modulators can be any suitable type of antisense nucleic acid molecule. “Classic” antisense molecules are typically short molecules, comprising gene-specific sequences that target particular RNA transcripts thereby preventing translation of the target RNA. Often such molecules are modified so that complexes formed between target transcripts and the antisense molecule is digested by RNase H or similar enzyme, thereby increasing the efficiency of the antisense molecule. Other antisense molecules are engineered to allow a ribozyme (e.g., RNase P) to destroy the target RNA in a similar efficiency-promoting sense (e.g., the antisense molecule can be associated with so-called “External Guide Sequences” (EGSs)). The Ln-5 modulator also can be a triple-helix-forming nucleic acids, which typically are gene-specific DNA molecules (whether such molecules are appropriately classified as antisense molecules is currently the subject of academic debate). These molecules can target a gene, e.g., a gene encoding a Ln-5-associated polypeptide that modifies Ln-5 in association with some stage of cancer progression and/or development of IBD. Anti-sense molecules can be designed to interact with a target nucleic acid molecule through either canonical or non-canonical base pairing. The interaction of the functional nucleic acid molecule and the target molecule is designed to promote the destruction of the target molecule through, for example, RNAseH mediated RNA-DNA hybrid degradation. Alternatively the functional nucleic acid molecule is designed to interrupt a processing function that normally would take place on the target molecule, such as transcription or replication. Antisense modulators can be designed based on the sequence of the target molecule. Numerous methods for optimization of binding efficiency by finding the most accessible regions of the target molecule exist. Exemplary methods would be in vitro selection experiments and DNA modification studies using DMS and DEPC. It is preferred that the antisense molecules bind the target molecule with a dissociation constant (k_{d}) of less than 10⁻⁶, more preferably less than 10⁻⁸, still more preferably less than 10⁻¹⁰, and yet more preferably less than 10⁻¹². A representative sample of methods and techniques which aid in the design and use of various antisense molecules can be found in the following non-limiting list of U.S. Pat. Nos. (anti-sense:) U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437; (aptamers:) U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698, (ribozymes:) U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Additionally, descriptions of relevant techniques specifically regarding siRNA can be found in the following literature: U.S. Pat. No. 6,506,559, International Publication No. WO 01/29058, International Publication No. WO 01/68836, International Publication No. WO 01/75164, U.S. Publication No. 20020114784, U.S. Publication No. 20030125281, U.S. Publication No. 2002162126, U.S. Publication No. 20030108923, U.S. Publication No. 20020173478, Fire, et al. Nature 391:806-811 (1998); Yang, et al., Mol. Cell. Biol. 21:7807-7816 (2001), Elbashir, et al., Nature 411:494-498 (2001), Hammond et al. Nat. Rev. Genet 2:110-119 (2001), and Sharp, Genes Dev. 15:485-490 (2001)).

In a particular aspect, the Ln-5 modulator is a siRNA (typically a ds RNA molecule of about 20-25 nucleotides) that interferes with the action of a target gene expression product, e.g., a gene expression product associated with a polypeptide that induces a modification in Ln-5 associated with some aspect of cancer progression and/or the development of IBD, ADPKD, or any of the other diseases or disorders described herein. The following DNA sequences correspond to exemplary siRNA sequences targeting the Ln-5 gamma 2 chain:

| | |
|---|---|
| AGTGCTCGATGTGACAACTCT | (SEQ ID NO:1) |
| AGCCAGATGCGACCGATGTCT | (SEQ ID NO:2) |
| AGTGTCCATAAGATCACCTCT | (SEQ ID NO:3) |
| AGCCTGGCAGAAAGTGAAGCT | (SEQ ID NO:4) |
| AGGAGATTGGGAGTCTGAACT | (SEQ ID NO:5) |

The following correspond to random siRNA sequences that can be used as controls:

| | |
|---|---|
| AGGGCCGACATAGATGAGACT | (SEQ ID NO:6) |
| AGCGTGGCTGAAAGTGAAGCT | (SEQ ID NO:7) |

Methods of Use

As mentioned above, Ln-5 modulators can induce, promote, and/or enhance physiological responses associated with changes in Ln-5 activity. These changes in physiological response or activity in turn are associated with the promotion and/or enhancement of treatment or prophylactic protection against various Ln-5-assocaited diseases and disorders.

Unless otherwise stated or clearly contradicted by context, the term “treatment” refers to the delivery of an effective amount of a therapeutically active compound of the invention with the purpose of preventing any symptoms or disease state to develop or with the purpose of easing, ameliorating, or eradicating (curing) such symptoms or disease states already developed. “Treatment” can also mean inducing, promoting, and/or enhancing one or more physiological responses or features associated with treatment of the disease state, or reducing the severity, spread, onset, or risk of developing a disease state, as described herein. The term “treatment” is thus meant to include prophylactic treatment. Terms similar to “treatment” (e.g., “treating”) should be similar construed unless otherwise stated or clearly contradicted. However, it will be understood that “therapeutic regimens” intended for an already established disease and such “prophylactic regimens” used for the prevention of disease, ameliorating the severity of a later-developed disease, increasing the time to onset of a disease condition, or combination of any thereof, etc., provided by the teachings herein, can be considered separate and independent aspects of this invention (e.g., such regimens may differ in terms of dosage, dosage regimen, etc.). As an example, prophylactic treatment or a treatment aimed at reducing the risk of a patient developing a certain disease state can be evaluated by comparing the incidence of the disease state in a treated subject group to the incidence of the disease state in an untreated control group during a predetermined study period.

An “effective amount” as defined herein is an amount of an active agent, such as an Ln-5 modulator, which, upon administration to a subject suffering from or at risk for a particular disease, disease state, or disorder, results in one or more physiological responses associated with treatment of the disease, disease state, or disorder, or in the relief or reduction of one or more disease symptoms in the subject. A “therapeutically effective amount” is an amount of an active agent which, upon administration to a subject suffering from a particular disease, disease state, or disorder, results in one or more physiological responses associated with treatment of symptoms of features of established disease. A “prophylactically effective amount” is an amount of an active agent that, upon administration to a subject, prevents or reduces the risk for the appearance of one or more disease symptoms in the subject. A “physiologically effective amount” herein refers to an amount of an active agent that induces, promotes, and/or enhances one or more physiological responses. In embodiments relating to combination treatments using an Ln-5 modulator and a secondary agent, “effective amounts” of the agents refers to amounts of the respective agents that are, separately or in combination, and upon administration to a subject suffering from or at risk for a particular disease, disease state, or disorder, results in one or more physiological responses associated with treatment of the disease, disease state, or disorder, or in the relief or reduction of one or more disease symptoms in the subject. In particular embodiments, the effective amounts have a combined efficacy or increased efficacy, or are a synergistically effective.

In one aspect, the invention provides a method of facilitating the prevention and/or treatment of inflammatory bowel disease (“IBD”). Thus, for example, the invention provides a method of inducing, promoting, and/or enhancing one or more physiological responses associated with the treatment or prevention of IBD comprising administering to a patient identified as having or being at significant risk of developing IBD a composition comprising a Ln-5 modulator (e.g., an Ln-5-associated antibody, an Ln-5 binding protein, or both) in an amount sufficient to detectably induce, promote, and/or enhance the one or more physiological responses.

Such methods of the invention can be used to induce, facilitate, promote, and/or enhance the treatment and/or prevention of any aspect of IBD. Thus, in one aspect, such methods of the invention are useful in the treatment and/or prevention of ulcerative colitis (UC). Such methods of the invention also or alternatively can be used in the treatment and/or prevention of Crohn's disease (CD).

In more particular aspects, the performance of such methods of the invention detectably results in the reduction of swelling, scarring, inflammation, or any combination thereof in gastrointestinal tissues, such as the tissues of the colon (in terms of spread and/or severity). The performance of such a method also or alternatively can be associated with, e.g., a reduction in the severity and/or spread of colitis, ileitis, and/or proctitis. In other aspects, practicing such methods can reduce the size and/or number of ulcers in the gastrointestinal system of a patient, such as in the lining of the colon of a patient afflicted with ulcerative colitis. Performing such methods also can be associated with a reduction of frequency of bowel movements (lessening diarrhea) and/or reducing the amount of blood in the stool of the patient/host. Such methods also can be associated with a reduction in abdominal pain. Furthermore the performance of such methods may decrease the risk of developing or reduce the severity of colorectal cancer. The performance of such inventive methods also or alternatively can be associated with the reduction in rectal bleeding and a reduction in the risk of colon rupture. Methods for assessing such effects are known in the art and discussed elsewhere herein (e.g., barium enema inspection and/or surgical inspection of the colon). An improvement in any of the aforementioned disease symptoms or characteristics can be a physiological response according to the invention.

As indicated above, another aspect of the invention is the discovery that inducing, enhancing, and/or promoting the treatment and/or prevention of IBD can be achieved by the administration of a therapeutically effective and/or prophylactically effective amount of a Ln-5 modulator, such as a Ln-5-associated antibody, a Ln-5 binding protein, or a combination of such peptides. The invention also provides a method of reducing the severity, spread, onset, or risk of developing IBD and/or promoting the treatment thereof, as well as decrease the risk of developing or decrease the severity of complications related to IBD, such as colorectal cancer, comprising administering to a patient identified as having or being at significant risk of developing IBD a composition comprising a Ln-5 modulator (e.g., an Ln-5-regulating siRNA, Ln-5-regulating antisense molecule, an Ln-5-associated antibody, an Ln-5 binding protein, or a combination of any thereof) in an amount sufficient to detectably reduce the severity, spread, onset, or risk of developing and/or promote the treatment of IBD in a population of similar patients. A prophylactic or therapeutic amount of such a composition can be determined, for example, in the context of clinical trials for IBD. Such studies and studies of patients with IBD can be used to assess the risk of developing IBD and/or average times for onset in similar patients. Clinical indicators for IBD that are relevant in this respect are well known and included, for example, degree of inflammation (as determined by e.g., by visualization through colonoscopy or sigmoidoscopy), anemia, white blood cell count, and degree of ulceration (as determined by, e.g., barium enema x-ray). Relevant indicators for CD can include the number and/or size of fistulas, perianal sores, bowel wall thickening, and/or the number and/or size of “skip lesions” (or simply “skips”—alternating areas of inflamed mucosa). Relevant indicators for ulcerative colitis can include inflammatory polyps, coarseness of granular mucosa, “lead pipe colon” (rigidity and symmetrical narrowing of the colon lumen), “burnt-out colon” (distensible colon without haustral markings—general loss of colonic haustrae is relevant), filiform polyposis, occult or frank blood loss, weight loss, neutrophil infiltration, loss of epithelial cells (potentially resulting in multiple ulcerations, fibrosis, dysplasia and longitudinal retraction of the colon), folate deficiency, and toxic megacolon. Other imaging procedures (as opposed to endoscopic and/or radiological procedures) can be used to assess IBD. Magnetic resonance spectroscopy (MRS) is a variant of magnetic resonance imaging (MRI) that may prove to be useful for assessing IBD. Computed tomography (CT) scans and ultrasound may be useful for determining the extent of the disease on the intestine and for detecting abscesses and other complications of advanced IBD. A reduction of any of the aforementioned indicators can be used as physiological response indicator according to the invention.

The invention also provides a method for detectably improving the quality of life of a patient suffering from IBD comprising administering a therapeutically effective amount of a composition comprising a Ln-5 modulator, such as a Ln-5-associated antibody, a Ln-5 binding protein, or a combination thereof, for a duration, under conditions, and in amount(s) sufficient to detectably improve the quality of life of the patient. The use of quality of life assessments in IBD treatment has been well characterized in the art. See, e.g., Han et al., Scand J Gastroenterol. 1998 September; 33(9):961-6; Guyatt et al., Gastroenterology. 1989 March; 96(3):804-10; Panwala et al., J Immunol. 1998 Nov. 15; 161(10):5733-44; Irvine et al., Gastroenterology. 1994 February; 106(2):287-96; Cheung et al., J Clin Epidemiol. 2000 Mar. 1; 53(3):297-306; Irvine, Scand J Gastroenterol Suppl. 1993; 199:36-9; Irvine et al., Am J Gastroenterol. 1996 August; 91(8):1571-8; Hjortswang et al., Scand J Gastroenterol. 2001 January; 36(1):77-85; Pallis, Dig Liver Dis. 2000 November; 32(8):682-8; Irvine, Scand J Gastroenterol Suppl. 1995; 208:136-40; Han et al., Am J Gastroenterol. 2000 January; 95(1):145-51; de Boer et al., Eur J Gastroenterol Hepatol. 1995 November; 7(11):1043-50; Bernklev et al., Scand J Gastroenterol. 2002 October; 37(10):1164-74; van der Eijk et al., Am J Gastroenterol. 2001 December; 96(12):3329-36; Konig et al., Eur J Gastroenterol Hepatol. 2002 November; 14(11):1205-15; Griffiths et al., J Pediatr Gastroenterol Nutr. 1999 April; 28(4):S46-52; Otley et al. J Pediatr Gastroenterol Nutr. 2002 October; 35(4):557-63; Int J Nurs Stud. 2002 August; 39(6):583-90; Xiao et al. Curr Gastroenterol Rep. 2002 December; 4(6):490-6; and Ferry J Pediatr Gastroenterol Nutr. 1999 April; 28(4):S15-8. Appropriate duration and dosage conditions can readily be determined by one of ordinary skill in the art using known principles, examples of which are discussed elsewhere herein.

The foregoing methods of preventing and/or treating IBD can further comprise a step of administering a second anti-IBD agent in association with (or in combination with) the composition comprising the Ln-5 modulator (e.g., a Ln-5-associated antibody, Ln-5 binding protein, or both can be combined with one or more secondary IBD ameliorating agents and/or IBD preventative agents). Any suitable secondary anti-IBD agent can be administered to the host or patient in any suitable manner. Examples of secondary anti-IBD agents include GLP-2, GLP-2 analogues, Kunitz-type inhibitors, trefoil factor 2 (TFF2) peptides (monomers or dimers), trefoil factor 1 (TFF1) peptides (monomers or dimers) and trefoil factor 3 (TFF3) peptides (monomers or dimers), non-steroidal anti-inflammatory drugs (NSAIDS), steroids or alcohol, tissue factor agonists, CD45 phosphatase inhibitor, 1,2,4-triazoles, alpha-amino ketones, aminosalicylates, such as Sulfasalazine (Azulfidine), corticosteroids, and immunosuppressive agents such as cyclosporine, prednisone, 6-mercaptopurine or azathioprine (converted in the liver to 6mercaptopurine), various leukotriene, PAF and cyclooxygenase pathway inhibitors, and antibodies or cloned receptor molecules which target specific proteins in the inflammatory cascade such as IL-1, IL-6 or TNF-α. Another approach to mitigating the oxidative stress resulting from an inflammatory response is to employ nitrone-related therapeutics (NRTs).

In a particular aspect, the prevention and/or treatment of IBD with a Ln-5-associated antibody, Ln-5 binding protein, and/or other Ln-5 modulator composition is enhanced by the associated administration of an agent selected from 5-ASA, anti-TNFα agents and antibodies (e.g., Enbrel, Remicade), an inhibitor of VCAM, and an inhibitor of ICAM.

Additional useful anti-IBD agents include TRA-fic (TRAFYK)-inhibiting agents, such as RDP58 (SanStat Medical Crp.), ICAM-1 inhibiting agents, such as the antisense nucleic acid agent alicaforsen (Isis Pharmaceuticals), interferon beta, infliximab, rosiglitazone, anti-IL-12 and anti-IL-6R antibodies, and daclizumab. Other useful secondary agents, anti-IBD methods, and methods related to the diagnosis of IBD and assessment of anti-IBD agents are described in, e.g., U.S. Pat. Nos. 6,610,674, 6,545,056, 6,479,465, 6,326,364, 6,265,374, 6,194,465, 6,183,951, 5,968,741, 5,368,854, 6,613,751, 6,599,509, 6,558,661, 6,551,593, 6,540,993, 6,500,418, 6,482,409, 6,395,273, 6,358,939, 6,291,441, 6,218,129, 6,214,373, 6,166,044, 6,143,785, 6,046,179, 6,036,978, 6,028,095, 6,025,387, 6,013,670, 5,948,818, 5,932,214, 5,928,883, 5,905,081, 5,888,969, 5,834,021, 5,776,972, 5,710,181, 5,674,754, and 5,599,795.

Any of the above-described IBD-related methods also can be combined with surgical techniques (e.g., ileostomy, ileoanal or ileorectal anastomosis (particularly for ulcerative colitis), continent ileostomy, proctocolectomy, resection and/or bypass, intestinal transplant, coletomy, ileoanal pullthrough, ileoanal “pouch” (e.g., J-pouch) techniques, stem cell/precursor cell and/or tissue implantation, and the like) and/or therapeutic nutritional programs (e.g., folate therapy, vitamin D therapy, anti-inflammatory vitamin C therapy, vitamin B12 therapy, zinc/calcium therapy, etc., or IBD-associated “neutraceutical” therapy) associated with the treatment and/or prevention of IBD. Relevant therapeutic methods and other matters related to IBD are described in, e.g., Hanauer and Bayless, Advanced Therapy Of Inflammatory Bowel Disease (2d ed. 2000), U.S. Pat. No. 6,486,349, and various references cited therein. The practice of such methods also can be combined with a therapeutic diet regimen, involving, e.g., fat reduction, lactose reduction, and/or fiber reduction.

The invention also provides methods for preventing, delaying the onset of, ameliorating, and/or treating IBD-associated cancers. Thus, for example, the invention provides a method of reducing the risk of developing cancer in a patient suffering from an inflammatory bowel disease comprising administering to the patient a composition comprising a Ln-5 modulator (e.g., an Ln-5-associated antibody, an Ln-5 binding protein, or both) in an amount sufficient to detectably reduce the risk of developing cancer in a population of similar patients. In a more general sense, the invention provides a method of inducing, promoting, and/or enhancing a physiological response associated with the prevention and/or treatment of cancer in a patient suffering from an inflammatory bowel disease and identified as having or at risk of developing a inflammatory bowel disease-associated cancer comprising administering to the patient a composition comprising a Ln-5 modulator (such as a Ln-5-modulating siRNA, an Ln-5-associated antibody, an Ln-5 binding protein, or a combination of any thereof) in an amount sufficient to detectably induce the physiological response.

Most colorectal cancers, regardless of etiology, arise from adenomatous polyps. Only adenomas are clearly premalignant, and only a minority of such lesions ever develop into cancer. Most polyps produce no symptoms and remain clinically undetected. Occult blood in the stool may be found in <5% of patients with such lesions. Clinically, the probability of an adenomatous polyp becoming a cancer depends on the gross appearance of the lesion, its histologic features, and its size. Adenomatous polyps may be pedunculated (stalked) or sessile (flat-based). Cancers develop more frequently in sessile polyps. Histologically, adenomatous polyps may be tubular, villous (i.e., papillary), or tubulovillous. Villous adenomas, most of which are sessile, become malignant more than three times as often as tubular adenomas. The likelihood that any polypoid lesion in the large bowel contains invasive cancer is related to the size of the polyp, being negligible (<2%) in lesions <1.5 cm, intermediate (2 to 10%) in lesions 1.5 to 2.5 cm in size, and substantial (10%) in lesions >2.5 cm. The invention provides a method of reducing the risk of developing cancer in such patients by the administration of a LN-5 modulator.

Additionally, where a patient has been identified as suffering from an IBD-associated cancer (e.g., by biopsy or other investigative procedure or technique that reveals cancerous and/or precancerous growth), an anti-cancer agent (e.g., a cancer cell targeted apoptotic agent, a tumor vaccine, an antisense molecule targeted to a tumor suppressor or oncogene, or an anti-angiogenesis agent growth factor and signal transduction modulators, cell cycle control, and immunomodulatory agents that enhance the immune response against tumor cells) can be administered in association with the Ln-5-associated antibody, Ln-5 binding protein, other Ln-5 modulator, or combination thereof. Exemplary anti-cancer agents are provided herein. Where a patient has been identified as being at substantial risk of developing an inflammatory bowel disease-associated cancer, the method can comprise administering a cancer preventative agent (e.g., a so-called tumor vaccine or an antisense nucleotide inhibitor of an oncogene) to the patient in association with the Ln-5-associated antibody, Ln-5 binding protein, other Ln-5 modulator or combination. Examples of anti-cancer therapeutics and cancer preventative agents are known in the art and examples of such agents are further described elsewhere herein. In a particular aspect, the method of treating and/or preventing an IBD-associated cancer comprises administering the composition to a population of cells having a higher than normal aneuploid DNA distribution. In another aspect, the method of treating and/or preventing an IBD-associated cancer comprises administering the composition to a person in whom Ln-5 expression is increased compared to a healthy individual.

Where a patient has been identified as suffering from an IBD-associated cancer, surgery can be performed on that patient in association with the administration of an Ln-5-associated antibody, Ln-5 binding protein, other Ln-5 modulator, or combination thereof. Surgery may be both surgery in primary management removing tumor or tumors, secondary cytoreductive surgery and palliative secondary surgery. The administration of with the Ln-5-associated antibody, Ln-5 binding protein, other Ln-5 modulator, or combination thereof can be carried out prior to surgery, during surgery, after surgery, or any suitable combination thereof.

In another aspect, the invention provides a method of reducing the risk of developing, preventing the development, or reducing the spread, growth, or spread and growth of renal cysts in a patient identified as having such a cyst or having a substantial risk of developing such a cyst comprising administering a composition comprising a Ln-5 modulator (such as an Ln-5-associated antibody, an Ln-5 binding protein, or both) to the patient in an amount sufficient to detectably reduce the risk of developing, preventing the development, or reducing the spread, growth, or spread and growth of renal cysts in a population of similar patients. Methods of identifying patients having or at risk of developing such cysts are known in the art (see, e.g., McHugh et al., Radiology. 1991 February; 178(2):383-5; King et al., Kidney Int. 2003 December; 64(6):2214-21; and Sutters et al., J Lab Clin Med. 2003 February; 141(2):91-101). For example, a genetic test has been developed and is commercially available to detect mutations in the PKD1 and PKD2 genes. This test can detect the presence of the autosomal dominant PKD mutations before cysts develop. The most common symptoms are pain in the back and the sides (between the ribs and hips), and headaches. The dull pain can be temporary or persistent, mild or severe. High blood pressure occurs early in the disease, often before cysts appear. In addition, diverticulosis (small sacs on the colon), kidney stones, liver and pancreatic cysts, hematuria (blood in the urine), urinary tract infections may occur. A reduction of any of these symptoms, and/or a reduction in renal cyst size or number, can be a physiological response according to the present invention.

Animal models of diseases associated with such cysts, such as ADKPD, are known. See, e.g., Thomson et al., Am J Physiol Renal Physiol. 2003 November; 285(5):F870-80 and Gattone et al. Nature Med. 2003 vol 9 no 10 page 1323-1326.

In yet another aspect, the invention provides a method of inducing, promoting, and/or enhancing a physiological response in a patient identified as having a renal cyst or having a substantial risk of developing a renal cyst comprising administering to a composition comprising an Ln-5-associated antibody, an Ln-5 binding protein, other Ln-5 modulator, or combination of any thereof (the phrases combination thereof and combination of any thereof may be used interchangeably throughout unless otherwise stated or contradicted by context) to the patient in an amount sufficient to detectably induce, promote, and/or enhance the physiological response.

In a further aspect, the invention provides a method of ameliorating a disease state associated with the growth and/or spread of renal cysts in a patient having such cysts comprising administering a composition comprising a Ln-5 modulator (e.g., an Ln-5-associated antibody, an Ln-5 binding protein, other Ln-5 modulator, or combination of any thereof), to the patient in an amount sufficient to detectably ameliorate the disease state. In this respect, the invention provides a method of reducing the motility and/or invasion of such cysts in a patient.

In methods related to modulating Ln-5 function in connection with renal cysts, the effectiveness of the Ln-5-associated antibody and/or Ln-5 binding protein may be enhanced by the associated administration (pre-administration, co-administration, and/or post administration) of anti-renal cyst agents/anti-Autosomal Dominant Polycystic Kidney Disease (ADPKD) agents and/or agents associated with the management of ADPKD. Examples of such agents include ACE inhibitors, calcium channel blockers, Trimethoprim Sulfamethoxazole, Chloramphenicol, and Ciprofloxacin, among others. Cyst penetrating antibiotics such as trimethoprim-sulfamethoxazole, ciprofloxacin, and chloramphenicol also can be useful for the treatment of ADPKD and associated disorders/diseases. Chronic pain from cysts also can be managed by cyst puncture and sclerosis with ethanol.

The effectiveness of ADPKD therapeutic and/or prophylactic techniques can be assessed by ultrasonography, intravenous urography, and nephrotomography, CT scanning, and MRI, as well as other techniques discussed herein.

Physiological conditions associated with ADPKD that may be modulated by administration of a Ln-5 modulator (e.g., a Ln-5-associated antibody and/or Ln-5 binding protein) composition include the reduction of kidney size from an enlarged state, reduction in the number and/or size of cysts, reduction in the number of hemorrhagic cysts, reduction in flank pain, increase in urine pH, and reduction of ureteral obstruction.

The invention further provides a method of reducing the risk of developing, delaying the onset of, reducing the severity, preventing and/or treating irritable bowel syndrome (“IBS”) in a patient suffering from IBS or identified as being of substantial risk of developing IBS comprising administering to the host a therapeutically or prophylactically effective amount of an Ln-5-associated antibody, an Ln-5 binding protein, other Ln-5 modulator, or a combination thereof to achieve the desired outcome(s). Patients with IBS basically fall into two broad clinical groups. Most commonly, patients have abdominal pain associated with altered bowel habits that consist of constipation, diarrhea, or both. In the second group, patients have painless diarrhea. Abdominal pain in IBS is highly variable in intensity and location; it is localized to the hypogastrium in 25%, the right side in 20%, to the left side in 20%, and the epigastrium in 10% of patients. It is frequently episodic and crampy, but it may be superimposed on a background of constant ache. Pain may be mild enough to be ignored or it may interfere with daily activities. However, patients with severe IBS often wake repeatedly during the night. Additionally, between 25 and 50% of patients with IBS complain of dyspepsia, heartburn, nausea, and vomiting. An improvement in any of these symptoms after administering an Ln-5 modulator according to the present invention is a physiological effect.

In yet another aspect, the invention provides a method of promoting, enhancing, and/or inducing one or more physiological effects in a patient associated with the prevention and/or treatment of irritable bowel syndrome comprising administering to the patient a composition comprising an amount of an Ln-5-associated antibody, an Ln-5 binding protein, or both or other Ln-5 modulator or combination of Ln-5 modulators in an amount sufficient to detectably promote, enhance, and/or induce the physiological effect(s). In another sense, the invention provides a method of enhancing the management of IBS (the same can be said of IBD and/or ADKPD) comprising regularly administering a composition comprising one or more Ln-5 modulators to achieve the desired therapeutic effect.

The assessment of treatment of IBS can be made on any suitable basis using any suitable criteria (e.g., quality of life). Appropriate factors for assessing IBS management/treatment are known and described in, e.g., Graef, AWHONN Lifelines. 2003 August-September; 7(4):324-30; Olden et al., Rev Gastroenterol Disord. 2003; 3 Suppl 3:S3-S11; Corazziari et al., Aliment Pharmacol Ther. 2003 Sep. 15; 18(6):569-80; Gordon et al., Dig Dis Sci. 2003 July; 48(7):1317-23. Somers et al., Gastroenterol Clin North Am. 2003 June; 32(2):507-29; Lacy et al., Arch Intern Med. 2003 Jun. 9; 163(11):1374-5; van Zanten, Rev Gastroenterol Disord. 2003; 3 Suppl 2:S12-7; and Ron, Isr Med Assoc J. 2003 March; 5(3):201-2.

The compositions of the invention can be useful in promoting the treatment and/or prevention of other gastrointestinal disorders, such as gastrointestinal endometriosis. The composition of the invention can be administered in association with application of IBS prevention, management, and/or treatment techniques, such as are described in Cash et al., Curr Gastroenterol Rep. 2003 December; 5(6):468-75; Huerta et al. Pharmacoepidemiol Drug Saf. 2003 October-November; 12(7):583-8; Lacy et al., Rev Gastroenterol Disord. 2003; 3 Suppl 3:S32-42; Gunn et al., Postgrad Med J. 2003 March; 79(929):154-8. Related methods of treating, preventing, assessing, and/or identifying IBS are described in, e.g., U.S. Pat. Nos. 6,653,339, 6,638,928, 6,596,759, 6,566,369, 6,562,629, 6,429,209 6,395,705 6,369,079 6,228,040 6,203,797 5,965,557 5,900,233 5,434,174 4,745,131, and 4,239,768.

Another feature of the invention involves methods of inducing, promoting, and/or enhancing the treatment and/or preventing of cancer by the administration of a composition comprising a combination of a Ln-5-associated antibody, Ln-5 binding protein, other Ln-5 modulator, or a combination thereof, and at least one second anti-cancer agent or cancer preventative agent, wherein the composition is associated with a synergistic, additive, or significantly superior effects or significantly reduced side effects in comparison with similar separate administration of such agents.

In one exemplary aspect, the invention provides a method of reducing cancer progression in a mammalian host afflicted with cancer comprising administering a composition comprising a synergistically effective combination of an Ln-5-associated antibody, an Ln-5 binding protein, or both, or other Ln-5 modulator or combination of Ln-5 modulators and one or more anti-cancer agents to the host in an amount sufficient to detectably reduce the progression of the cancer and/or time to relapse for the patient.

In another exemplary aspect, the invention provides a method of increasing the ratio of quiescent to invasive neoplastic cells in a mammalian host comprising administering a therapeutically effective amount of a composition comprising a synergistically effective combination of a Ln-5 modulator (e.g., an Ln-5-associated antibody, an Ln-5 binding protein, or both) and one or more anti-cancer agents to the host so as to increase the ratio of quiescent to invasive cells in the host. Tumor biopsy material or other tumor tissue can be used in such a detection where BrdU or Ki67 can be used to as proliferation marker and laminin-5 expression or matrix metalloproteinase secretion as a marker for invasiveness.

In a further aspect, the invention provides a method of preventing the formation of cancer associated tubular networks in a mammalian host comprising administering a therapeutically effective amount of a composition comprising a synergistically effective combination of a Ln-5 modulator (e.g., an Ln-5-associated antibody, an Ln-5 binding protein, or both) and one or more anti-cancer agents to the host so as to detectably reduce the risk of developing cancer associated tubular networks, prolong the onset of cancer associated tubular networks, and/or reduce the number of expected cancer associated tubular networks formed in the host.

In yet another aspect, the invention provides a method of reducing the invasive potential of cancer cells in a mammalian host comprising administering a therapeutically effective amount of a composition comprising a synergistically effective combination of a Ln-5 modulator (e.g., an Ln-5-associated antibody, an Ln-5 binding protein, or both) and one or more anti-cancer agents to the host so as to detectably reduce the invasive potential of the cancer cells.

In still another aspect, the invention provides a method of reducing cell migration, reducing tumor growth, reducing neoplastic or pre-neoplastic cell division, or any combination thereof in a mammalian host comprising administering a therapeutically effective amount of a composition comprising a synergistically effective combination of an Ln-5-associated antibody, an Ln-5 binding protein, other Ln-5 modulator, or combination thereof and one or more anti-cancer agents to the host so as to achieve the desired outcome(s).

In a further aspect, the invention provides a method of promoting remission of a cancer comprising administering a composition comprising a synergistic combination of a Ln-5-associated antibody, a Ln-5 binding protein, other Ln-5 modulator, or combination thereof and a second anti-cancer agent.

In yet another aspect, the invention provides a method for modulating MAP kinase activity in neoplastic or preneoplastic cells of a mammalian host comprising contacting the cells with a physiologically effective amount of a composition comprising a synergistically effective combination of an Ln-5-associated antibody, an Ln-5 binding protein, other Ln-5 modulator, or combination thereof and one or more anti-cancer agents to the cells so as to detectably modulate MAP kinase activity in the cells.

In an even further aspect, the invention provides a method for reducing the risk of developing a cancer, reducing the time to onset of a cancerous condition, reducing the severity of a cancer diagnosed in the early stages, and/or reducing the affected area of a cancer upon development thereof in a mammalian host, comprising administering to a host a prophylactically effective amount of a composition comprising a synergistically effective combination of an Ln-5-associated antibody, an Ln-5 binding protein, other Ln-5 modulator, or combination thereof and one or more anti-cancer prophylactic agents so as to achieve the desired physiological effect(s).

A cancer cell in the context of this invention is any cell that divides and reproduces abnormally with uncontrolled growth (e.g., by exceeding the “Hayflick limit” of normal cell growth (as described in, e.g., Hayflick, Exp. Cell Res., 37,614 (1965)). “Cancer progression,” as used herein, refers to any event or combination of events that promote, or which are indicative of, the transition of a normal, non-neoplastic cell to a cancerous, neoplastic cell. Examples of such events include phenotypic cellular changes associated with the transformation of a normal, non-neoplastic cell to a recognized pre-neoplastic phenotype, and cellular phenotypic changes that indicate transformation of a pre-neoplastic cell to a neoplastic cell.

Aspects of cancer progression (also referred to herein as “cancer progression stages”) include cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, aneuploidy, and focus formation), proliferation of transformed cells, development of metastatic potential, migration and metastasis (e.g., the disassociation of the cell from a location and relocation to another site), new colony formation, tumor formation, tumor growth, neotumorogenesis (formation of new tumors at a location distinguishable and not in contact with the source of the transformed cell (s)), and any combinations thereof.

The methods of the present invention can be used to reduce, treat, prevent, or otherwise ameliorate any suitable aspect of cancer progression. The methods of the invention are particularly useful in the reduction and/or amelioration of tumor growth and metastatic potential, as described further herein. Methods that reduce, prevent, or otherwise ameliorate such aspects of cancer progression are preferred. A particularly preferred aspect of the invention is the reduction of the metastatic and/or invasive (aggressive) potential of cancer cells. Another favorable aspect is the effectiveness of such methods in the treatment of cancers characterized by micrometastases.

The detection of cancer progression can be achieved by any suitable technique, several examples of which are known in the art. Examples of suitable techniques include PCR and RT-PCR (e.g., of cancer cell associated genes or “markers”), biopsy, electron microscopy, positron emission tomography (PET), computed tomography, immunoscintigraphy and other scintegraphic techniques, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques. Examples of these and other suitable techniques are described in, e.g., Rieber et al., Cancer Res., 36 (10), 3568-73 (1976), Brinkley et al., Tex. Rep. Biol. Med., 37, 26-44 (1978), Baky et al., Anal. Quant. Cytol., 2 (3), 175-85 (1980), Laurence et al., Cancer Metastasis Rev., 2 (4), 351-74 (1983), Cooke et al., Gut, 25 (7), 748-55 (1984), Kim et al, Yonsei Med. J., 26 (2), 167-74 (1985), Glaves, Prog. Clin. Biol. Res., 212, 151-67 (1986), McCoy et al., Itnmunol. Ser., 53, 171-87 (1990), Jacobsson et al., Med. Oncol. Tumor. Pharmacother., 8 (4), 253-60 (1991), Swierenga et al., IARC Sci. Publ., 165-93 (1992), Hirnle, Lymphology, 27 (3), 111-3 (1994), Laferte et al., J. Cell Biochem., 57 (1), 101-19 (1995), Machiels et al., Eur. J. Cell Biochem., 66 (3), 282-92 (1995), Chaiwun et al., Pathology (Phila), 4 (1), 155-68 (1996), Jacobson et al, Ann. Oncol., 6 (Suppl. 3), S3-8 (1996), Meijer et al., Eur. J. Cancer, 31A (7-8), 1210-11 (1995), Greenman et al., J. Clin. Endocrinol. Metab., 81 (4), 1628-33 (1996), Ogunbiyi et al., Ann. Surg. Oncol., 4 (8), 613-20 (1997), Merritt et al., Arch. Otolaryngol. Head Neck Surg., 123 (2), 149-52 (1997), Bobardieri et al., Q. J. Nucl. Med., 42 (1), 54-65 (1998), Giordano et al., J. Cell Biochem, 70 (1), 1-7 (1998), Siziopikou et al., Breast J., 5 (4), 221-29 (1999), Rasper, Surgery, 126 (5), 827-8 (1999), von Knebel et al., Cancer Metastasis Rev., 18 (1), 43-64 (1999), Britton et al., Recent Results Cancer Res., 157, 3-11 (2000), Caraway et al., Cancer, 90 (2), 126-32 (2000), Castillo et al., Am. J. Neuroadiol., 21 (5), 948-53 (2000), Chin et al., Mayo Clin. Proc., 75 (8), 796-801 (2000), Kau et al., J. Ortohinolaryngol. Relat. Spe., 62 (4), 199-203 (2000), Krag, Cancer J. Sci. Am., 6 (Suppl. 2), S121-24 (2000), Pantel et al., Curr. Opin. Oncol., 12 (1), 95-101 (2000), Cook et al., Q. J. Nucl. Med., 45 (1), 47-52 (2001), Gambhir et al., Clin. Nucl. Med., 26 (10), 883-4 (2001), MacManus et al., Int. J. Radiat. Oncol. Biol. Phys., 50 (2), 287-93 (2001), Olilla et al., Cancer Control., 8 (5), 407-14 (2001), Taback et al., Recent Results Cancer Res., 158, 78-92 (2001), and references cited therein. Related techniques are described in U.S. Pat. Nos. 6,294,343, 6,245,501, 6,242,186, 6,235,486, 6,232,086, 6,228,596, 6,200,765, 6,187,536, 6,080,584, 6,066,449, 6,027,905, 5,989,815, 5,939,258, 5,882,627, 5,829,437, 5,677,125, and 5,455,159 and International Patent Applications WO 01/69199, WO 01/64110, WO 01/60237, WO 01/53835, WO 01/48477, WO 01/04353, WO 98/12564, WO 97/32009, WO 97/09925, and WO 96/15456.

A reduction of cancer progression can be any detectable decrease in (1) the rate of normal cells transforming to neoplastic cells (or any aspect thereof), (2) the rate of proliferation of pre-neoplastic or neoplastic cells, (3) the number of cells exhibiting a pre-neoplastic and/or neoplastic phenotype, (4) the physical area of a cell media (e.g., a cell culture, tissue, or organ (e.g., an organ in a mammalian host)) comprising pre-neoplastic and/or neoplastic cells, (5) the probability that normal cells will transform to neoplastic cells, (6) the probability that cancer cells will progress to the next aspect of cancer progression (e.g., a reduction in metastatic potential), or (7) any combination thereof. Such changes can be detected using any of the above-described techniques or suitable counterparts thereof known in the art, which are applied at a suitable time prior to the administration of the composition of the invention. Times and conditions for assaying whether a reduction in cancer potential has occurred will depend on several factors including the type of cancer, type and amount of Ln-5-associated antibody and/or Ln-5 binding protein and/or other Ln-5 modulator. The ordinarily skilled artisan will be able to make appropriate determinations of times and conditions for performing such assays applying techniques and principles known in the art and/or routine experimentation.

The methods of the invention can be used to reduce the cancer progression of any suitable type of cancer. Advantageously, the methods of the invention can be used to reduce the cancer progression in prostate cancer cells, melanoma cells (e.g., cutaneous melanoma cells, ocular melanoma cells, and lymph node-associated melanoma cells), breast cancer cells, colon cancer cells, and lung cancer cells. The methods of the invention can be used to reduce cancer progression in both tumorigenic and non-tumorigenic cancers (e.g., non-tumor-forming hematopoietic cancers). The methods of the invention are particularly useful in the treatment of epithelial cancers and/or colorectal cancers, breast cancers, lung cancers, vaginal cancers, cervical cancers, and/or squamous cell carcinomas (e.g., of the head and neck).

Additional features of such methods include the reduction in the size and/or number of and/or prevention of the formation of tubular networks associated with Ln-5. Another feature of the invention is a method of preventing poorly aggressive neoplastic cells from developing a vasculogenic phenotype. Such aspects can be combined with the general feature of reducing the invasive potential (aggressiveness) and/or metastatic potential of cancers by such methods. Reduction of neoplastic and/or preneoplastic cell migration, reduction of cell division, and/or reduction of cell migration by administration of compositions of the invention are additional features.

Anti-cancer and cancer preventative agents include immunomodulating agents such as IL-2, IL-21, TGF-beta inhibitors, inhibiting killer inhibitory receptors (KIRs), anti CD20 like Rituximab, anti-CD33 like Gemtuzumab, anti-CD52 like Alemtuzumab, IL19, IL20, tumor vaccines, interferons, (anti-)TNF-alpha, anti-IL-2R, anti-IL-15R, telomerase inhibitors, “cross-linking” agents such as platinum agents (cisplatin, carboplatin, etc.), antisense oligonucleotides or siRNA reducing the expression of oncogenes, mutated or deregulated genes, and immunomodulatory (oligo-) nucleotides. Non-limiting examples of additional anti-cancer therapeutic and/or prophylactic agents and therapies fluoropyrimidine carbamates, such as capecitabine; non-polyglutamatable thymidylate synthase inhibitors; nucleoside analogs, such as tocladesine; antifolates such as pemetrexed disodium; taxanes and taxane analogs; topoisomerase inhibitors; polyamine analogs; mTOR inhibitors (e.g., rapamcyin ester); alkylating agents (e.g., oxaliplatin); lectin inhibitors; vitamin D analogs (such as seocalcitol); carbohydrate processing inhibitors; antimetabolism folate antagonists; thumidylate synthase inhibitors; other antimetabolites (e.g., raltitrexed); ribonuclease reductase inhibitors; dioxolate nucleoside analogs; thimylate syntase inhibitors; gonadotropin-releasing hormone (GRNH) peptides; human chorionic gonadotropin; chemically modified tetracyclines (e.g., CMT-3; COL-3); cytosine deaminase; thymopentin; DTIC; carmustine; carboplatin; vinblastine; temozolomide; vindesine; thymosin-α; histone deacetylase inhibitors (e.g., phenylbutyrate); DNA repair agents (e.g., DNA repair enzymes and related compositions such as Dimericine™ (T4 endonuclease V-containing liposome)); gastrin peptides (and related compositions such as Gastrimmune™); GMK and related compounds/compositions (see, e.g., Knutson, Curr Opin Investig Drugs. 2002 January; 3(1):159-64 and Chapman et al., Clin Cancer Res. 2000 December; 6(12):4658-62); beta-catenin blockers/inhibitors and/or agents that lower the amount of beta-catenin production in preneoplastic or neoplastic cell nuclei (see, e.g., U.S. Pat. No. 6,677,116), agents that upregulate E-cadherin expression (or E-cadherin); agents that reduce slug (beta-catenin-associated) gene expression; agents that block, inhibit, or antagonize PAI-1 or that otherwise modulate urokinase plasminogen activator (uPA) interaction with the uPA receptor; survivins; DNA demethylating agents; “cross-linking” agents such as platinum-related anti-cancer agents (cisplatin, carboplatin, etc.); agents that block antiapoptotic signaling, such as agents that inhibit MAPK and Ras signaling pathways or components thereof (e.g., agents that interfere with the production and/or function of cyclin D); growth suppressive agents, such as an antimetabolite such as Cepecitabine/Xeloda, cytarabine/Ara-C, Cladribine/Leustatin, Fludaraine/Fludara, fluorouracil/5-FU, gemcitabine/Gemzar, mercaptopurine/6-MP, methotrexate/MTX, thioguanine/6-TG, Allopurinol/Zyloprim, etc.; an acylating agent such as Busulfan, Cyclophosphamide, mechlorethamaine, Melphalan, thiotepa, semustine, carboplatin, cisplatin, procarbazine, dacarbazine, Althretamine, Lomustine, Carmustine, Chlorambucil, etc.; a topoisomerase inhibitor such as Camptothecins as Topotecan, Irinotecan; such as Podophyllotoxins as Etoposide/VP16, Teniposide/VP26, etc.; an inhibitor of microtuble and/or spindle formation, such as Vincristine, Vinblastine, Vinorelbine, or Taxane such as Paxlitaxel, Docetaxel, combrestatin, Epothilone B, etc; RRR-alpha-tocopheryl succinate; anthracyclins as Daunorubicin/Cerubidine and Doxorubicin; idarubicin; mitomycins; plicamycin; retinoic acid analogues such as all trans retinoic acid, 13-cis retinoic acid, etc.; inhibitors of receptor tyrosine kinases; inhibitors of ErbB-1/EGFR such as iressa, Erbitux, etc.; inhibitors of ErbB-2/Her2 such as Herceptin, etc.; inhibitors of c-kit such as Gleevec; inhibitors of VEGF receptors such as ZD6474, SU6668, etc.; Inhibitors of ErbB3, ErbB4, IGF-R, insulin receptor, PDGFRa, PDGFRbeta, Flk2, Flt4, FGFR1, FGFR2, FGFR3, FGFR4, TRKA, TRKC, c-met, Ron, Sea, Tie, Tie2, Eph, Ret, Ros, Alk, LTK, PTK7, etc.; cancer related enzyme inhibitors such as metalloproteinase inhibitors such as marimastat, Neovastat, etc.; cathepsin B; modulators of cathepsin D dehydrogenase activity; glutathione-5-transferases and related compounds such as glutacylcysteine synthetase and lactate dehydrogenase; proteasome inhibitors (e.g., Bortezomib); tyrosine kinase inhibitors; farnesyl transferase inhibitors; HSP90 inhibitors (e.g., 17-allyl amino geld-anamycin) and other heat shock protein-inhibitors; mycophenolate mofetil; mycophenolic acid; asparaginase; calcineurin-inhibitors; TOR-inhibitors; multikine molecules; enkephalins (see, e.g., U.S. Pat. No. 6,737,397); SU11248 (Pfizer); BAY 43-9006 (Bayer and Onyx); inhibitors of “lymphocyte homing” mechanisms such as FTY720; Tarceva; Iressa; Glivec; thalidomide; and adhesion molecule inhibitors (e.g., anti-LFA, etc.). Additional anti-neoplastic agents that can be used in the combination composition and combination administration methods of the invention include those described in, e.g., U.S. Pat. Nos. 6,660,309, 6,664,377, 6,677,328, 6,680,342, 6,683,059, and 6,680,306, as well as International Patent Application WO 2003070921.

Where appropriate, one or more of such agents also or alternatively can be conjugated to a Ln-5 modulator. Such conjugates are another feature of the invention.

In one aspect, the invention provides combination compositions and combination delivery/administration protocols that include one or more biological response modifiers (BRMs) in addition to one or more Ln-5 modulators. BRMs generally are products produced by cells that stimulate or restore the ability of the immune system to act against disease agents (e.g., cancer cells), such as cytokines or antibodies.

In yet another aspect, an Ln-5 modulator and/or related compound is delivered in association with a molecule that binds to free Ln-5 γ2 and/or γ2 fragment peptides, such as a Ln-5-interacting integrin or γ2 peptide-binding fragment thereof (examples of such agents are known in the art).

Combination compositions and combination delivery methods also or alternatively can include anti-anergic agents (e.g., small molecule compounds, proteins, glycoproteins, or antibodies that break tolerance to tumor and cancer antigens).

Additional exemplary and particular types of compositions and therapies that may be used/applied with delivery of one or more Ln-5 modulators or related compositions are described in turn below. It will be recognized that these classifications of agents and methods are non-limiting and only used for convenience. Some of the previously mentioned agents may fall into one or more of the categories described below. Some anti-cancer agents also cannot be readily classified. This includes, for example, the relatively new tyrosine kinase inhibitor imatinib mesylate (Gleevec® or Glivec®). Another example of novel anti-cancer agents are agents that induce fusion of tumor cells, such as measles glycoproteins and related nucleic acids (see, e.g., U.S. Pat. No. 6,750,206).

In a particular aspect, the secondary anti-cancer agent is selected from one of the following agents: an alkylating agent such as Melphalan, Busulfan, Cis-platin, Carboplatin, Cyclophosphamide, Ifosphamide, Dacarbazine, Chlorambucil, Thiotepa, Lomustine, or Temozolamide, an anti-metabolite agent such as Cytarabine, Azathioprine, Fludarabine, phosphate, Fludarabine, 5-fluorouracil, Gemcitabine, Hydroxyurea, Cladribine, Thioguanine, Methotrexate, or Capecitabine; an anti-mitotic agent such as Vinorelbine, Vincristine, Vinblastine, Docetaxel, or Paclitaxel; a topoisomerase inhibitor such as Doxorubicin, Amsacrine, Irinotecan, Daunorubicin, Epirubicin, Mitomycin, Mitoxantrone, Idarubicin, Teniposide, Etoposide, or Topotecan; an antibiotic such as Actinomycin or Bleomycin; aspariginase; and a hormone or anti-hormone agent such as Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin, or Goserelin; as well as combinations of any of the aforementioned compounds.

One feature of the invention is embodied in compositions comprising at least one Ln-5 modulator and a suitable secondary antibody (e.g., a suitable secondary anti-cancer mAb). In general, such a composition can comprise any secondary antibody or combination of secondary antibodies that does not significantly interfere with the specificity, selectivity, and/or affinity of the Ln-5 modulator. Typically, a secondary antibody component of a Ln-5 modulator combination composition exhibits no more than about 5%, such as no more than about 10%, and commonly no more than about 20% competition for Ln-5 binding with the Ln-5 modulator of the composition.

In a particular aspect, the invention provides a combination composition that includes at least one Ln-5 modulator and at least one secondary anti-cancer monoclonal antibody. A number of suitable anti-cancer mAbs are known in the art and similar suitable antibodies can be developed against cancer-associated targets. Particular examples of suitable second anti-cancer mAbs include anti-CD20 mAbs (such as Rituximab and HuMax-CD20), anti-Her2 mAbs (e.g., Trastuzumab), anti-CD52 mAbs (e.g., Alemtuzumab and Capath® 1H), anti-EGFR mAbs (e.g., Cetuximab, HuMax-EGFr, and ABX-EGF), Zamyl, Pertuzumab, anti-A33 antibodies (see U.S. Pat. No. 6,652,853), anti-oncofetal protein mAbs (see U.S. Pat. No. 5,688,505), anti-PSMA mAbs (see, e.g., U.S. Pat. No. 6,649,163 and Milowsky et al., J Clin Oncol. 2004 Jul. 1; 22(13):2522-31. Epub 2004 Jun. 01), anti-TAG-72 antibodies (see U.S. Pat. No. 6,207,815), anti-aminophospholipid antibodies (see U.S. Pat. No. 6,406,693), anti-neurotrophin antibodies (U.S. Pat. No. 6,548,062), anti-C3b(i) antibodies (see U.S. Pat. No. 6,572,856), anti-cytokeratin (CK) mAbs, anti-MN antibodies (see, e.g., U.S. Pat. No. 6,051,226), anti-mts1 mAbs (see, e.g., U.S. Pat. No. 6,638,504), anti-PSA antibodies (see, e.g., Donn et al., Andrologia. 1990; 22 Suppl 1:44-55; Sinha et al., Anat Rec. 1996 August; 245(4):652-61; and Katzenwadel et al., Anticancer Res. 2000 May-June; 20(3A):1551-5); antibodies against CA125; antibodies against integrins like integrin beta1; antibodies/inhibitors of VCAM; anti-alpha-v/beta-3 integrin mAbs; anti-kininostatin mAbs; anti-aspartyl (asparaginyl) beta-hydroxylase (HAAH) intrabodies (see, e.g., U.S. Pat. No. 6,783,758); anti-CD3 mAbs (see, e.g., U.S. Pat. Nos. 6,706,265 and 6,750,325) and anti-CD3 bispecific antibodies (e.g., anti-CD3/Ep-CAM, anti-CD3/her2, and anti-CD3/EGP-2 antibodies—see, e.g., Kroesen et al., Cancer Immunol Immunother. 1997 November-December; 45(3-4):203-6); and anti-VEGF mAbs (e.g., bevacizumab). Other possibly suitable second mAb molecules include alemtuzumab, edrecolomab, tositumomab, ibritumomab tiuxetan, and gemtuzumab ozogamicin. In one aspect, the invention provides combination compositions and combination therapies that comprise one or more antibodies, typically monoclonal antibodies, targeted against angiogenic factors and/or their receptors, such as VEGF, bFGF, and angiopoietin-1; and monoclonal antibodies against other relevant targets (see also, generally, Reisfeld et al., Int Arch Allergy Immunol. 2004 March; 133(3):295-304; Mousa et al., Curr Pharm Des. 2004; 10(1):1-9; Shibuya, Nippon Yakurigaku Zasshi. 2003 December; 122(6):498-503; Zhang et al., Mol Biotechnol. 2003 October; 25(2):185-200; Kiselev et al., Biochemistry (Mosc). 2003 May; 68(5):497-513; Shepherd, Lung Cancer. 2003 August; 41 Suppl 1:S63-72; O'Reilly, Methods Mol. Biol. 2003; 223:599-634; Zhu et al., Curr Cancer Drug Targets. 2002 June; 2(2):135-56; and International Patent Application WO 2004/035537).

Where appropriate such antibodies can be conjugated to a cytotoxin, radionuclide, or another anti-cancer agent. Also where appropriate, immunogenic peptide targets of such antibodies can be used to induce an immune response in a combination composition or combination administration method of the invention.

Where appropriate the targets for these secondary antibodies also can be targeted by multispecific LN-5 modulators of the invention. Thus, for example, VH, VL, and/or CDR sequences from such mAbs also can be used in the context of bispecific and multispecific Ln-5 modulators, such as L5G2D3BPs, described elsewhere herein. Likewise, mAbs specific for targets discussed above with respect to bispecific mAbs, cancer antigens, and other molecules discuss with respect to other aspects also can be used in the methods of the invention and incorporated in the compositions of the invention.

Other antibodies developed against lymphomas, leukemia cells, micrometastases, and solid tumors also may be useful in combination methods and/or combination compositions of the invention. Antibodies that inhibit functions vital for tumor cell survival, growth, invasiveness and/or migration; antibodies that induce ADCC or CDC against tumor/cancer cells; antibodies that interrupt key cancer progression-related signaling events; and/or that deliver a toxic payload to preneoplastic and/or neoplastic cells can be particularly useful in such methods and compositions. Death of the tumor cells also might lead to the release of tumor antigens that “vaccinate” the immune system and stimulates it to produce a secondary response that then targets tumor/cancer cells. Thus, in one aspect, the invention provides a method of targeting a particular population of cancer cells or tumor(s) followed by monitoring of the patient for a secondary response, and providing further anti-cancer therapy if such secondary response is deemed unsatisfactory. Over-expressed oncogenes and tumor-specific antigens can be advantageous targets for such antibodies. Tumor antigens, which can be useful in this context or in their own right as immunogenic peptides (“vaccines”) are described in, for example, Stauss et al.: Tumor antigens recognized by T cells and antibodies. Taylor and Frances (2003) and Durrant et al., Expert Opin. Emerging Drugs 8(2):489-500 (2003).

In a further aspect, the invention provides a combination treatment therapy comprising such a combination of an Ln-5 modulator and one or more anti-cancer secondary mAb(s). It will be understood that variants of such secondary mAbs, derivatives of such secondary mAbs or secondary mAb variants, and functional fragments of any thereof, also or alternatively can be used in the methods of the invention and incorporated into the compositions of the invention.

In another aspect, the invention provides combination compositions and combination therapy methods involving a “chemotherapeutic agent” in addition to an anti-cancer Ln-5 modulator. Chemotherapeutic agents in the context of this invention typically refer to small molecule compounds selectively toxic to cancer and pre-cancer cells and/or exert one or more effects on the cell cycle of at least some cell types that likely include cancer and pre-cancer cells.

Combination of chemotherapeutic agents, which commonly are used in cancer treatment, also can be combined with one or more Ln-5 modulators, in composition or by co-delivery. For example, in one aspect the invention provides a method of treating cancer comprising delivering to a patient an effective amount of one or more Ln-5 modulators in association with an effective application of CHOP chemotherapy (chemotherapy comprising a combination of cyclophosphamide, doxorubicin, vincristine, and prednisone), such that an at least additive anti-cancer effect (if not a greater than additive effect) is obtained.

In another facet, a Ln-5 modulator and/or related composition is delivered in association with a chemotherapeutic that acts on the DNA level of cancer progression (non-limiting examples of conventional chemotherapeutic agents at the DNA level include anti-metabolites (such as 6-mercaptopurine, 6-thioguanine, methotrexate, 5-fluorouracil, and hydroxyurea), cytarabine, alkylating agents, procarbazine, topoisomerase inhibitors, platinum derivatives, anthracyclines, and antibiotics)).

In another particular aspect, the Ln-5 modulator or related composition is delivered to a subject or comprised in a composition with an “RNA level” chemotherapeutic agent (or combination thereof), nonlimiting examples of which include L-asparaginase, Vinca alkaloid, taxanes, anti-cancer taxane combination compositions (such as docetaxel-plus-prednisone), and topoisomerase inhibitors.

In another particular aspect, an Ln-5 modulator and/or related composition is delivered in association with an effective dose of dacarbazine (DTIC).

In another particular facet, the invention provides combination compositions and combination therapies that involve effective amount(s) of one or more Ln-5 modulators and one or more chemotherapeutic agents selected from 5-Fluorouracil, actinomycin D (Dactinomycin), amsacrine, arsenic trioxide, asparaginase, azadcytadine (5-azacytidine, 5AZ), busulfan (myleran), capecitabine, carboplatin (paraplatin), carmustine (BiCNU), chlorambucil (Leukeran), cisplatin (Platinol), cyclophosphamide (Cyroxan), cytarabine (Ara-C), Dacarbazine, Dactinomycin, Daunorubicin (Cerubidine), Docetaxel (Taxotere), Doxorubicin (Adriamycin Doxil), Epirubicin (Ellence), Etoposide (VP-16, Vespid), Fludarabine, Fluorouracil, Gemcitabine, Gleevac (Imatinib mesylate, STI 571), Hydroxyurea, Idarubicin, Ifosfamide (Ifex), Irinotecan, Liposomal Doxurubicin, Lomustine, Mechlorechamine (Mustargen), Melphalan, Mercaptopurine (6 MP), Methotrexate, Methyl CCNU, Mitomycin (Mutamycin), Mitoxantrone, Nitrogen Mustard, Nitrosoureas, Oxaliplatin, Paclitaxel (Taxol), Pentostatin, Plicamycin, Procarbazine, Streptozocin, Telcyta (alone or in combination with Doxil), Teniposide (Vumon), Thiotepa, Tretinoin, Vinblastine (Velban), Vincristine (Oncovin), and Vinorelbine (Navelbine).

To better illuminate the invention, non-limiting groups of chemotherapeutic agents useful in Ln-5 modulator combination compositions and therapies are described here.

In one aspect, the invention provides combination compositions and combination therapies characterized in comprising one or more alkylating agents. Alkylating agents are so named because of their ability to add alkyl groups to many electronegative groups under conditions present in cells. These agents stop tumor growth by cross-linking guanine bases in DNA double-helix strands—directly attacking DNA. This makes the strands unable to uncoil and separate. As this is necessary in DNA replication, cells affected by such agents can no longer divide. Examples of alkylating agents include Busulfan (Myleran), Busulfan Injection (Busulfex Injection), Carboplatin (Paraplatin), Carmustine Injection (BiCNU Injection), Chlorambucil (Leukeran), Cyclophosphamide Injection (Cytoxan Injection, Neosar), Dacarbazine (DTIC, DTIC-Dome), Ifosfamide (Ifex), Lomustine (CCNU, CeeNU), Mechlorethamine (Mustargen, Nitrogen mustard), Melphalan (Alkeran, L-PAM), Melphalan Injection (Alkeran Injection), Streptozocin (Zanosar), and Thiotepa (Thioplex).

In another facet, the invention provides combination compositions and therapies that comprise one or more Ln-5 modulators and one or more antimetabolites. Antimetabolites prevent cancer cells from processing nutrients and other substances that are necessary for normal activity in the cancer cells. More particularly, antimetabolites masquerade as purine or pyrimidine, which become the building blocks of DNA. They prevent these substances becoming incorporated in to DNA during the “S” phase of the cell cycle, thereby preventing cell division. There are several different cellular targets for antimetabolites. Some common classes of antimetabolites are folate antagonists, purine antagonists, and pyrimidine antagonists.

Folate antagonists, also known as antifolates, inhibit dihydrofolate reductase (DHFR), an enzyme involved in the formation of nucleotides. When this enzyme is blocked, nucleotides are not formed, disrupting DNA replication and cell division. Methotrexate is the primary folate antagonist used as a chemotherapeutic agent.

The purine antagonists function by inhibiting DNA synthesis in two different ways. They can inhibit the production of the purine containing nucleotides, adenine and guanine. If a cell doesn't have sufficient amounts of purines, DNA synthesis is halted and the cell cannot divide. They also may be incorporated into the DNA molecule during DNA synthesis. The presence of the inhibitor is thought to interfere with further cell division. Examples of purine antagonists include 6-Mercaptopurine, Dacarbazine, and Fludarabine.

Pyrimidine antagonists also can be combined or co-delivered with one or more Ln-5 modulators. Pyrimidine antagonists act to block the synthesis of pyrimidine containing nucleotides. These drugs typically have structures similar to the natural compound that they replace. By acting as ‘decoys’, these drugs can prevent the production of the finished nucleotides. They may exert their effects at different steps in that pathway and may directly inhibit crucial enzymes. Pyrimidine antagonists may also be incorporated into a growing DNA chain and lead to termination of the process. Examples of pyrimidine antagonists include 5-fluorouracil, Arabinosylcytosine, Capecitabine, and Gemcitabine.

In an exemplary aspect, the invention provides combination compositions and therapies characterized by including one or more metabolites selected from Floxuridine (FUDR, Fluorodeoxyuridine), Fludarabine Phosphate (Fludara), Gemcitabine Hydrochloride (Gemzar), Hydroxyurea (Droxia, Hydrea), Mercaptopurine (6-MP, Purinethol), Methotrexate (Rheumatrex, Trexall), Methotrexate Injection (Amethopterin, MTX Injection), Thioguanine (6-TG, TG), and combinations of any thereof.

The invention also relates to combination compositions and therapies including one or more antineoplastic hormones. Antineoplastic hormones interfere at the cellular level with receptors for growth stimulating proteins. By blocking the receptor, the production or release of growth factors is reduced. Examples of such agents include Diethylstilbestrol Injection (Stilphostrol Injection), Megestrol (Megace), and Mitotane (Lysodren).

In a further facet, the invention provides combination compositions and combination delivery methods characterized in including one or more mitotic inhibitors. Mitotic inhibitors generally prevent cell division by interfering with the protein called tubulin. Tubulin is the basic building block of the fibers that are responsible for ensuring that each cell continues to multiply. Examples of such agents include Docetaxel (Taxotere), Etoposide Injection (Toposar, VePesid Injection), Etoposide Oral (VP-16, VePesid Oral), Paclitaxel (Onxol, Taxol), Vinblastine (Velban), and Vincristine (Oncovin, Vincasar).

Another somewhat related class of classic chemotherapeutic agents is plant alkaloids. These alkaloids are derived from plants and block cell division by preventing microtubules being synthesized. These are vital for cell division and without them it can not occur. The main examples are vinca alkaloids such as vincristine.

Another also somewhat related class of classic chemotherapeutic agents is “genotoxic drugs.” Genotoxic drugs are chemotherapy agents that affect nucleic acids and alter their function. These drugs may directly bind to DNA or they may indirectly lead to DNA damage by affecting enzymes involved in DNA replication and possibly apoptosis. Such genotoxic chemotherapy treatments include alkylating agents, intercalating agents (drugs that “wedge” into the spaces between the nucleotides in the DNA double helix, thereby interfering with transcription and replication, and often inducing mutations), and enzyme inhibitors (e.g., agents that inhibit key enzymes, such as topoisomerases, involved in DNA replication inducing DNA damage). Thus, the genotoxic drug class of chemotherapeutic agents overlaps with other classes described elsewhere herein. A goal of treatment with any of these agents includes (i.e., a common mechanism of action associated with such agents is) the induction of DNA damage in the cancer cells. DNA damage, if severe enough, will induce cells to undergo apoptosis. Genotoxic chemotherapy drugs typically affect both normal and cancerous cells. The selectivity of the drug action is based on the sensitivity of rapidly dividing cells, such as cancer cells, to treatments that damage DNA. Examples of agents that can be classified as genotoxic agents include Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolamide, and Topotecan.

Another feature of the invention is embodied in combination methods and compositions that comprise one or more Ln-5 modulators (or delivery thereof) and one or more nucleic acids that act as anti-cancer agents (or delivery thereof).

In a particular aspect, the invention provides combination compositions and methods, wherein a Ln-5 modulator is combined with or delivered in association with a nucleic acid comprising a sequence encoding a tumor suppressor. In one exemplary facet, one or more Ln-5 modulators are delivered in association with the delivery of a p53 tumor suppressor gene (see, e.g., Roth et al., Oncology (Huntingt). 1999 October; 13(10 Suppl 5):148-54) and Nielsen et al., Cancer Gene Ther. 1998 January-February; 5(1):52-63). Additional tumor suppressor targets include, for example, BRCA1, RB1, BRCA2, DPC4 (Smad4), p21, E2F-1, FUS1 compounds (e.g., INGN 401), MSH2, MLH1, and DCC. Such a nucleic acid can be delivered in the form of a suitable vector, host cell, etc. For example, one or more Ln-5 modulators can be combined with or delivered in association with a replication-deficient adenovirus encoding human recombinant wild-type p53/SCH58500.

A further feature of the invention is the provision of combination methods and combination compositions that include one or more Ln-5 modulators and one or more nucleic acids that are able to reduce one or more aspects of expression of particular cancer-associated genes. Such agents include antisense nucleic acids and inhibitory RNA (iRNA) molecules.

In one exemplary aspect, the invention provides combination compositions and methods that involve one or more antisense nucleic acids targeted to oncogenes, mutated, or deregulated genes. In another exemplary aspect, the invention provides combination compositions and methods that involve at least one siRNA molecule targeted to mutated or deregulated genes. Another feature of the invention is combination compositions and methods that include one or more antisense oligonucleotides and/or siRNAs that reduce the expression of oncogenes or other cancer progression-related genes (e.g., Ln-5-targeted or integrin-targeted antisense molecules—which are described in, e.g., US Patent Application 2003224993 and O'Toole et al., Exp Cell Res. 1997 Jun. 15; 233(2):330-9, and/or antisense molecules against Ln-5-modulators, such as MT1-MMP antisense oligonucleotides (see, e.g., Giles et al., J Cell Sci. 2001 August; 114(Pt 16):2967-76)), other inhibitors of Ln-5 production or activity (e.g., p300—see, e.g., Miller et al., J Biol. Chem. 2000 Mar. 17; 275(11):8176-82), mutated genes, and/or deregulated genes.

For example, a Ln-5 modulator, such as a L5G2D3BP, can be combined with or administered in association with an anti-cancer antisense nucleic acid (e.g., Genasense™ (augmerosen/G3139)), LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCA, clusterin, IGFBPs, protein kinase A, cyclin D1, or Bcl-2—see, e.g., Benimetskaya et al., Clin Prostate Cancer. 2002 June; 1(1):20-30; Tortora et al., Ann N Y Acad. Sci. 2003 December; 1002:236-43; Gleave et al., Ann N Y Acad. Sci. 2003 December; 1002:95-104; Lahn et al., Ann N Y Acad. Sci. 2003 December; 1002:263-70; Kim et al., Int J Oncol. 2004 January; 24(1):5-17; Stahel et al., Lung Cancer. 2003 August; 41 Suppl 1:S81-8; Stephens et al., Curr Opin Mol Ther. 2003 April; 5(2):118-22; Cho-Chung, Arch Pharm Res. 2003 March; 26(3):183-91; and Chen, Methods Mol Med. 2003; 75:621-36)). In another exemplary aspect, a Ln-5 modulator is delivered in association with or combined in a composition with an anti-cancer inhibitory RNA molecule (see, e.g., Lin et al., Curr Cancer Drug Targets. 2001 November; 1(3):241-7, Erratum in: Curr Cancer Drug Targets. 2003 June; 3(3):237; Lima et al., Cancer Gene Ther. 2004 May; 11(5):309-16; Grzmil et al., Int J Oncol. 2004 January; 24(1):97-105; Collis et al., Int J Radiat Oncol Biol Phys. 2003 Oct. 1; 57(2 Suppl):S144; Yang et al., Oncogene. 2003 Aug. 28; 22(36):5694-701; and Zhang et al., Biochem Biophys Res Commun. 2003 Apr. 18; 303(4):1169-78 for discussion relating to such iRNA molecules, related principles, and related methods).

In another facet, the invention provides combination compositions and combination administration methods where a L5G2D3BP is combined with an anti-cancer nucleozyme, such as a ribozyme, examples of which include angiozyme (Ribozyme Pharmaceuticals) (see e.g., Pennati et al., Oncogene. 2004 Jan. 15; 23(2):386-94; Tong et al., Clin Lung Cancer. 2001 February; 2(3):220-6; Kijima et al., Int J Oncol. 2004 March; 24(3):559-64; Tong et al., Chin Med J (Engl). 2003 October; 116(10):1515-8; and Orlandi et al., Prostate. 2003 Feb. 1; 54(2):133-43) and herzyme (in a related sense see U.S. Pat. No. 6,617,438). Additional anti-cancer ribozymes are described in, e.g., US Patent Applications 20030195164, 20030050236, and 20030105043 and U.S. Pat. Nos. 6,482,803 and 6,489,163. See also Poliseno et al., Current Pharmaceutical Biotechnology August 2004, vol. 5, no. 4, pp. 361-368(8) for a review of RNA-based drugs.

In yet another aspect, a Ln-5 modulator is combined or co-delivered with an immunostimulatory nucleic acid (in another aspect, a nucleic acid comprising a sequence encoding a Ln-5 modulator and at least one immunostimulatory sequence is provided). Numerous examples of suitable immunostimulatory nucleic acids have been described in the art (see, e.g., Krieg, Trends in Microbiol 7: 64-65 (1999); Wooldridge et al., Curr Opin Oncol. 2003 November; 15(6):440-5; Jahrsdorfer et al., Semin Oncol. 2003 August; 30(4):476-82; Jahrsdorfer et al., Curr Opin Investig Drugs. 2003 June; 4(6):686-90; Carpentier et al., Front Biosci. 2003 Jan. 1; 8:e115-27; U.S. Pat. No. 6,406,705; U.S. Pat. No. 6,218,371; US Patent Application 20040181045; and US Patent Application 20040087538).

In another aspect, the invention provides methods for treating Ln-5 associated conditions that comprising delivering one or more Ln-5 modulators and/or combining one or more Ln-5 modulators (or related compositions) with at least one Ln-5-encoding nucleic acid (in a form associated with normal cell basement membrane attachment/association), at least one nucleic acid that upregulates endogenous Ln-5 production (e.g., by so-called gene activation), and/or one or more cells expressing Ln-5 at levels at least as great as in normal basement membrane-associated epithelial cells. Other functional gene replacement methods also can be used in the context of the methods and reflected in compositions of this invention (e.g., providing a nucleic acid encoding a non-cancer-associated version of a tumor suppressor such as p53). Another use of gene therapy is the introduction of enzymes into these cells that make cancer cells susceptible to particular chemotherapy agents (e.g., introducing thymidine kinase into cancer cells so as to make them susceptible to aciclovir).

In a further aspect, the invention provides combination compositions and administration methods wherein a Ln-5 modulator is combined with or co-administered with a basal lamina-targeted and/or basal lamina-associated factor modulating anti-cancer molecules (e.g., a molecule that inhibits breakdown of the basal lamina in cancer progression), such as ginsenoside-Rb2, anti-MMP-1 antibodies, anti-integrin antibodies, anti-MMP2 antibodies and inhibitors, anti-MT1-MMP antibodies and inhibitors, anti-EGF-R antibodies, anti-BMP-1 inhibitors and antibodies, and inhibitors of urokinase-type plasminogen activator (uPA) and/or plasminogen activation to plasmin (aprotinin, amiloride, EACA, tranexamic acid, anti-uPA antibody). In another aspect, the invention provides such compositions and methods wherein the composition or method comprises an inhibitor of Thymosin beta 4.

In an additional facet, the invention provides combination compositions and combination administration methods wherein a Ln-5 modulator is combined or codelivered with a virus or related molecule (e.g., a virus like particle, a viral nucleic acid, etc.) that acts as an active agent against cancer. In one aspect, the invention provides combination compositions and methods that comprise one or more Ln-5 modulators (or related compositions) and at least one oncolytic virus. Examples of such viruses include oncolytic adenoviruses and herpes viruses, which may or may not be modified viruses (see, e.g., Teshigahara et al., J Surg Oncol. 2004 January; 85(1):42-7; Stiles et al., Surgery. 2003 August; 134(2):357-64; Zwiebel et al., Semin Oncol. 2001 August; 28(4):336-43; Varghese et al., Cancer Gene Ther. 2002 December; 9(12):967-78; and Wildner et al., Cancer Res. 1999 Jan. 15; 59(2):410-3).

Various viruses, viral proteins, and the like can be used in combination compositions and combination administration methods. Replication-deficient viruses, that generally are capable of one or only a few rounds of replication in vivo, and that are targeted to tumor cells, can, for example, be useful components of such compositions and methods. Such viral agents can comprise or be associated with nucleic acids encoding immunostimulants, such as GM-CSF and/or IL-2. Both naturally oncolytic and such recombinant oncolytic viruses (e.g., HSV-1 viruses; reoviruses; replication-deficient and replication-sensitive adenovirus; etc.) can be useful components of such methods and compositions (see, e.g., Varghese et al., Cancer Gene Ther. 2002 December; 9(12):967-78; Zwiebel et al., Semin Oncol. 2001 August; 28(4):336-43; Sunarmura et al., Pancreas. 2004 April; 28(3):326-9; Shah et al., J Neurooncol. 2003 December; 65(3):203-26; and Yamanaka, Int J Oncol. 2004 April; 24(4):919-23).

Additional features of the invention include combination administration methods and combination compositions wherein a Ln-5 modulator is combined or delivered with an anti-cancer immunogen, such as a cancer antigen/tumor-associated antigen (e.g., an epithelial cell adhesion molecule (Ep-CAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, CEA, cyclin-dependent kinase 4, β-catenin, capsase-B, tyrosinase, cancer-associated viral vaccines (e.g., human papillomavirus vaccines), tumor-derived heat shock proteins, and the like, additional examples of which are described elsewhere herein) (see also, e.g., Acres et al., Curr Opin Mol Ther 2004 Feb. 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). A number of other suitable cancer antigens/tumor-associated antigens described herein (e.g., gp75) and similar molecules known in the art also or alternatively can be used in such combination administration methods or incorporated in such combination compositions.

Anti-cancer immunogenic peptides also include anti-idiotypic “vaccines” such as BEC2 anti-idiotypic mAb (Mitumomab—see, e.g., Chapman, Curr Opin Investig Drugs. 2003 June; 4(6):710-5 and McCaffery et al., Clin Cancer Res. 1996 April; 2(4):679-86), CeaVace and related anti-idiotypic mAbs (see, e.g., Foon et al., J Clin Oncol. 1999 September; 17(9):2889-5), anti-idiotypic mAb to MG7 mAb (see, e.g., Fengtian et al., Chin Med Sci J. 2002 December; 17(4):215-9), and other anti-cancer anti-idiotypic Abs (see, e.g., Birebent et al., Vaccine. 2003 Apr. 2; 21(15):1601-12, Li et al., Chin Med J (Engl). 2001 September; 114(9):962-6, Schmitt et al., Hybridoma. 1994 October; 13(5):389-96, Maloney et al., Hybridoma. 1985 Fall; 4(3):191-209, Raychardhuri et al., J Immunol. 1986 Sep. 1; 137(5):1743-9, Pohl et al., Int J Cancer. 1992 Apr. 1; 50(6):958-67, Bohlen et al., Cytokines Mol Ther. 1996 December; 2(4):231-8, and Maruyama, J Immunol Methods. 2002 Jun. 1; 264(1-2):121-33). Such anti-idiotypic Abs can be advantageously optionally conjugated to a carrier, which may be a synthetic (typically inert) molecule carrier, a protein (e.g., keyhole limpet hemocyanin (KLH) (see, e.g., Ochi et al., Eur J Immunol. 1987 November; 17(11):1645-8)), or a cell (e.g., a red blood cell—see, e.g., Wi et al., J Immunol Methods. 1989 Sep. 1; 122(2):227-34)).

Compositions and combination administration methods of the invention also include the inclusion or coadministration of nucleic acid vaccines, such as naked DNA vaccines encoding such cancer antigens/tumor-associated antigens (see, e.g., U.S. Pat. Nos. 5,589,466, 5,593,972, 5,703,057, 5,879,687, 6,235,523, and 6,387,888).

In another aspect, the combination administration method and/or combination composition comprises an autologous vaccine composition. In a further aspect, the combination composition and/or combination administration method comprises a whole cell vaccine or cytokine-expressing cell (e.g., a recombinant IL-2 expressing fibroblast, recombinant cytokine-expressing dendritic cell, and the like) (see, e.g., Kowalczyk et al., Acta Biochim Pol. 2003; 50(3):613-24; Reilly et al., Methods Mol Med. 2002; 69:233-57; and Tirapu et al., Curr Gene Ther. 2002 February; 2(1):79-89). Another example of a therapeutic autologous cell method that can be useful in combination methods of this invention is the MyVax® Personalized Immunotherapy method (previously referred to as GTOP-99) (available through Genitope Corporation—Redwood City, Calif., USA) (see U.S. Pat. Nos. 5,972,334 and 5,776,746).

In a further aspect, combination compositions and/or combination administration methods of the invention comprise administration of an immunomodulatory compound or modulator thereof (e.g., an anti-inhibitory immunomodulatory antibody). Examples of such compounds include T cell activating and proliferation-promoting molecules, such as B7 molecules (B7-1, B7-2, variants thereof, and fragments thereof) (see, e.g., Adv Exp Med Biol. 2000; 465:381-90 and US Patent Application 20030208058), ICOS (inducible co-stimulator) molecules, and OX40 molecules (see Coyle et al., Springer Semin Immunopathol. 2004 February; 25(3-4):349-59 and 6,312,700). Another example of such a molecule is an inhibitor of a negative T cell regulator, such as an antibody against CTLA4, such as MDX-010 (Phan et al. (2003) Proc. Natl. Acad. Sci. U.S.A. 100: 8372). Antibodies against several members of the TNF receptor (TNFR) family have been shown to augment T cell proliferative responses. Antibodies to CD27 have also been shown to enhance T cell proliferation. The interaction of the integrin family member LFA-1 (lymphocyte function-associated antigen 1, or CD18/CD11a) with its ligands intercellular adhesion molecule (ICAM)-1, -2 and -3 is well known to be an important participant in the activation of T cells. SLAM (signaling lymphocyte activation molecule, or CDw150) is another T cell regulator. The heat-stable antigen (HSA or CD24) is a glycophosphatidylinositol (GPI)-linked protein of 27 amino acids found on the surface of hematopoietic and neuronal cells in an extensively glycosylated 38-70 kDa form that enhances T cell proliferation. 4-1 BB is a co-stimulatory receptor for T cells. TNFR-associated factors (TRAFs) also are T cell signaling molecules. CD40L also can act as an immunomodulator. The CD2-LFA3 pathway also is important to T cell regulation (and accordingly agents that act on it can be included in combination methods and compositions). NK cell activating and proliferating agents, such as stimulatory KIR molecules also can be included in such combination methods and compositions. Other immunomodulating agents that can also or alternatively be included in such combination compositions and methods are TGF-beta inhibitors.

Cytokines and chemokines, which represent an important subset of immunomodulators, are discussed in detail in the following discussion.

The invention provides combination composition and combination delivery methods comprising at least one Ln-5 modulator and at least one anti-cancer cytokine, chemokine, or combination thereof.

In general, any suitable anti-cancer cytokine and/or chemokine can be used with and/or combined with Ln-5 modulators in the methods and compositions of this invention. Suitable chemokines and cytokines result in a detectably greater and/or more comprehensive immune response to cancer cells or related tissues (e.g., tumors) in vivo and do not substantially impede the binding of the L5G2D3BP(s) in the composition/method.

Examples of suitable cytokines and growth factors include interferons (e.g., IFNβ, IFNα (e.g., INFα2b), and IFNγ (e.g., IFNγ1b)) and interleukins (e.g., IL-2, IL-4, IL-6, IL-7, IL-10, IL-12, IL-13, IL-15, IL-18, IL-23, IL-24, IL-27, IL-28a, IL-28b, IL-29, etc.). Additional cytokines that can be included in such compositions and methods include KGF, IFNα, GM-CSF, CD40L, Flt3 ligand, stem cell factor, ancestim, and TNFα (see, e.g., Dranoff, Nat Rev Cancer. 2004 January; 4(1):11-22 and Szlosarek, Novartis Found Symp. 2004; 256:227-37; discussion 237-40, 259-69).

Suitable chemokines can include Glu-Leu-Arg (ELR)-negative chemokines such as IP-10, MCP-3, MIG, and SDF-1 alpha from the human CXC and C—C chemokine families. Suitable cytokines include cytokine derivatives, cytokine variants, cytokine fragments, and cytokine fusion proteins (see, e.g., Eliason, BioDrugs, 2001; 15(11):705-11 (with respect to PEGylated cytokines) and Shibuya et al., Laryngoscope. 2003 November; 113(11):1870-84 (other cytokine derivatives), and WO 01/79258 (albumin-cytokine fusion proteins)).

These and other methods involving naturally occurring peptide-encoding nucleic acids herein can alternatively or additionally performed by “gene activation” and homologous recombination gene upregulation techniques, such as are described in U.S. Pat. Nos. 5,968,502, 6,063,630, and 6,187,305 and European Patent Publication 0 505 500. Additionally useful cytokines for such combination therapies and compositions are described elsewhere herein.

In another aspect, the invention provides a combination composition or combination administration method comprising an Ln-5 modulator and an adjuvant, typically in further combination with an anti-cancer immunogenic peptide (or surrogate nucleic acid/nucleic acid-encoding molecule). Non-limiting examples of suitable adjuvants are QS21, GM-CSF, SRL-172, histamine dihydrochloride, thymocartin, Tio-TEPA, monophosphoryl-lipid A/micobacteria compositions, alum, incomplete Freund's Adjuvant, Montanide ISA, Ribi Adjuvant System, TiterMax adjuvant, syntex adjuvant formulations, immune-stimulating complexes (ISCOMs), GerbuR adjuvant, CpG oligodeoxynucleotides, lipopolysaccharide, and polyinosinic:polycytidylic acid.

Combination compositions and combination administration methods also can involve “whole cell” and “adoptive” immunotherapy methods and “internal vaccination” techniques. For example, such methods can comprise infusion or re-infusion of immune system cells (e.g., tumor-infiltrating lymphocytes (TILs), such as CD4+ and/or CD8+ T cells (e.g., T cells expanded with tumor-specific antigens and/or genetic enhancements), antibody-expressing B cells or other antibody producing/presenting cells, dendritic cells (e.g., anti-cytokine expressing recombinant dendritic cells, dendritic cells cultured with a DC-expanding agent such as GM-CSF and/or Flt3-L, and/or tumor-associated antigen-loaded dendritic cells), anti-tumor NK cells, so-called hybrid cells, or combinations thereof (see, e.g., Fishman et al., Expert Rev Anticancer Ther. 2003 December; 3(6):837-49; Whiteside et al., Cancer Immunol Immunother. 2004 March; 53(3):240-8; Conrad et al., Curr Opin Mol Ther. 2003 August; 5(4):405-12; Trefzer et al., Mol Biotechnol. 2003 September; 25(1):63-9; Reinhard et al., Br J Cancer. 2002 May 20; 86(10):1529-33; Korbelik et al., Int J Cancer. 2001 Jul. 15; 93(2):269-74; Costa et al., J Immunol. 2001 Aug. 15; 167(4):2379-87; Hanson et al., Immunity. 2000 August; 13(2):265-76; Matsui et al., Int Immunol. 2003 July; 15(7):797-805; and Ho et al., Cancer Cell. 2003 May; 3(5):431-7). Cell lysates also may be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see, e.g., Bystryn et al., Clinical Cancer Research Vol. 7, 1882-1887, July 2001), optionally admixed with adjuvants such as alum, also can be advantageous components in such methods and methods. U.S. Pat. No. 6,699,483 provides another example of a whole cell anti-cancer therapy. Additional examples of such whole cell immunotherapies that can be usefully combined in Ln-5 modulator-related compositions and methods are described elsewhere herein.

In another aspect, one or more Ln-5 modulators are delivered to a patient in association with the delivery of an effective amount of antigen-pulsed dendritic cells or other anti-cancer immune cells (e.g., NK cells).

In yet another aspect, a Ln-5 modulator can be delivered to a patient in combination with the application of an internal vaccination method. Internal vaccination refers to induced tumor or cancer cell death, such as drug-induced or radiation-induced cell death of tumor cells, in a patient, that typically leads to elicitation of an immune response directed towards (i) the tumor cells as a whole or (ii) parts of the tumor cells including (a) secreted proteins, glycoproteins or other products, (b) membrane-associated proteins or glycoproteins or other components associated with or inserted in membranes, and/or (c) intracellular proteins or other intracellular components. An internal vaccination-induced immune response may be humoral (i.e. antibody—complement-mediated) or cell-mediated (e.g., the development and/or increase of endogenous cytotoxic T lymphocytes that recognize the internally killed tumor cells or parts thereof). In addition to radiotherapy, non-limiting examples of drugs and agents that can be used to induce said tumor cell-death induction and internal vaccination methods include conventional chemotherapeutic agents, cell-cycle inhibitors, anti-angiogenesis drugs, monoclonal antibodies, apoptosis-inducing agents, and signal transduction inhibitors.

In another aspect, the invention provides combination compositions and combination administration methods that involve at least one Ln-5 modulator and one or more cell cycle control/apoptosis regulators (or cell cycle/apoptosis “regulating agents”).

A cell cycle control/apoptosis regulator that can be combined with Ln-5 modulator(s) can include, for example, one or more molecules that target and modulate cell cycle control/apoptosis regulators such as (i) cdc-25 (with NSC 663284 as a non-limiting example (see, e.g., Pu et al (2003) J Biol Chem 278, 46877)), (ii) cyclin-dependent kinases that overstimulate the cell cycle (non-limiting examples of which are flavopiridol (L868275, HMR1275; Aventis), 7-hydroxystaurosporine (UCN-01, KW-2401; Kyowa Hakko Kogyo), and roscovitine (R-roscovitine, CYC202; Cyclacel)—as reviewed by Fischer & Gianella-Borradori (2003) Exp Op Invest Drugs 12, 955-970), and (iii) telomerase modulators (such as BIBR1532 (Damm et al (2001) EMBO J 20, 6958-6968) and SOT-095 (Tauchi et al (2003) Oncogene 22, 5338-5347)). Mycobacterium DNA has been reported to be capable of inducing apoptosis in cancer cells (see, e.g., U.S. Pat. No. 6,794,368).

Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2 (see Igney and Krammer (2002) Nature Rev. Cancer 2, 277-288; Makin; Dive (2003) Trends Mol Med 9, 2519; Smyth et al (2003) Immunity 18, 1-6; and Panaretakis et al. (2003) Oncogene 22, 4543-4556).

In another aspect, the invention provides combination compositions and combination delivery methods comprising a telomerase inhibitor, telomerase vaccine, or combination thereof in addition to at least one Ln-5 modulator or related molecule. Examples of such compositions and related techniques are described in U.S. Pat. Nos. 6,440,735 and 6,713,055.

In yet another aspect, the invention provides combination compositions and combination administration methods that comprise one or more growth factor inhibitors.

A number of antibodies (e.g., mAbs) against growth factors and growth factor receptors are known that can be useful in promoting the treatment of cancer. For example, antibodies against the extracellular ligand binding domain of epidermal growth factor receptor (EGF-R) proteins that are abnormally activated in epithelial tumors can be useful in the treatment of aggressive epithelial cell-derived tumors. Antibodies against low molecular weight molecules and small molecules that inhibit the tyrosine kinase domains of such receptors also can be useful in combination compositions or combination administration methods. Non-limiting examples of such molecules include Herceptin (monoclonal antibody), Cetuximab (monoclonal antibody), Tarceva (small molecule low molecular weight inhibitor), and Iressa (small molecule low molecular weight inhibitor). Additional related and useful antibodies suitable for inclusion in such combination compositions and administration methods are described elsewhere herein.

In a further aspect, the invention provides combination compositions and methods that include one or more Ln-5 modulators (or related molecule surrogates) and one or more inhibitors of angiogenesis, neovascularization, and/or other vascularization (such agents are referred to by terms such as anti-angoigenesis agents, anti-angiogenic drugs, etc. herein). Nonlimiting examples of such agents include (individually or in combination) endostatin and angiostatin (reviewed in Marx (2003) Science 301, 452-454) and derivatives/analogues thereof; anti-angiogenic heparin derivatives and related molecules (e.g., heperinase III); VEGF-R kinase inhibitors and other anti-angiogenic tyrosine kinase inhibitors (e.g., SU011248—see Rosen et al., Clinical Oncology; May 31-Jun. 3, 2003, Chicago, Ill., USA (abstract 765)); temozolomide; Neovastat™ (Gingras et al., Invest New Drugs. 2004 January; 22(1):17-26); Angiozyme™ (Weng et al., Curr Oncol Rep. 2001 March; 3(2):141-6); NK4 (Matsumoto et al., Cancer Sci. 2003 April; 94(4):321-7); macrophage migration inhibitory factor (MIF); cyclooxygenase-2 inhibitors; resveratrol (see, e.g., Sala et al., Drugs Exp Clin Res. 2003; 29(5-6):263-9); PTK787/ZK 222584 (see, e.g., Klem, Clin Colorectal Cancer. 2003 November; 3(3):147-9 and Zips et al., Anticancer Res. 2003 September-October; 23(5A):3869-76); anti-angiogenic soy isoflavones (e.g., Genistein—see, e.g., Sarkar and Li, Cancer Invest. 2003; 21 (5):744-57); Oltipraz; thalidomide and thalidomide analogs (e.g., CC-5013—see, e.g., Tohnya et al., Clin Prostate Cancer. 2004 March; 2(4):241-3); other endothelial cell inhibitors (e.g., Squalamine and 2-methoxyestradiol); fumagillin and analogs thereof; somatostatin analogues; pentosan polysulfate; tecogalan sodium; molecules that block matrix breakdown (such as suramin and analogs thereof (see, e.g., Marchetti et al., Int J Cancer. 2003 Mar. 20; 104(2):167-74, Meyers et al., J Surg Res. 2000 Jun. 15; 91(2):130-4, Kruger and Figg, Clin Cancer Res. 2001 July; 7(7):1867-72, and Gradishar et al., Oncology. 2000 May; 58(4):324-33)); dalteparin (Scheinowitz et al., Cardiovasc Drugs Ther. 2002 July; 16(4):303-9); matrix metalloproteinase inhibitors (such as BMS-275291—see Rundhaug, Clin Cancer Res. 2003 February; 9(2):551-4; see generally, Coussens et al. Science 2002; 295:2387-2392); angiocol; anti-PDGF mAbs and other PDGF (platelet derived growth factor) inhibitors; and PEDFs (pigment epithelium derived growth factors).

In another aspect, the invention provides combination compositions and combination administration methods wherein at least one Ln-5 modulator is combined with or delivered in association with a hormonal regulating agent, such as an anti-androgen and/or anti-estrogen therapy agent or regimen (see, e.g., Trachtenberg, Can J Urol. 1997 June; 4(2 Supp 1):61-64; Ho, J Cell Biochem. 2004 Feb. 15; 91(3):491-503), tamoxifen, a progestin, a luteinizing hormone-releasing hormone (or an analog thereof or other LHRH agonist), or an aromatase inhibitor (see, e.g., Dreicer et al., Cancer Invest. 1992; 10(1):27-41). Steroids (often dexamethasone) can inhibit tumour growth or the associated edema (brain tumors) and also can be suitable for combination with Ln-5 modulators (or related compound surrogates thereof). One or more Ln-5 modulators can be similar provided or combined with an antiandrogene such as Flutaminde/Eulexin; a progestin, such as hydroxyprogesterone caproate, Medroxyprogesterone/Provera, Megestrol acepate/Megace, etc.; an adrenocorticosteroid such as hydrocortisone, prednisone, etc.; a luteinising hormone-releasing hormone (LHRH) analogue such as buserelin, goserelin, etc.; and/or a hormone inhibitor such as octreotide/Sandostatin, etc. In a particular aspect, Ln-5 modulator(s) are provided or combined with an anti-cancer agent that is an estrogen receptor modulator (ERM) such as tamoxifen, idoxifene, fulvestrant, droloxifene, toremifene, raloxifene, diethylstilbestrol, ethinyl estradiol/Estinyl, etc., or a combination of any thereof. Combination compositions and combination administration methods also or alternatively can comprise tamoxifen. Further teachings relevant to cancer immunotherapy are provided in, e.g., Berczi et al., “Combination Immunotherapy of Cancer” in NEUROIMMUNE BIOLOGY, Volume 1: New foundation of Biology, Berczi I, Gorczynski R, Editors, Elsevier, 2001;pp. 417-432.

In a particular aspect, one or more Ln-5 modulators (or suitable related molecule surrogates therefor—such substitution of Ln-5 modulators with surrogates is contemplated throughout unless otherwise stated or clearly contradicted) is combined with or delivered in association with one or more aromatase inhibitors, such as anastrazole/Arimidex, aminoglutethimide/Cytraden, Exemestane, etc. Anti-aromatase agents inhibit the cytochrome P-450 component of the aromatase enzyme complex by interfering with the electron transfer from NADPH. Examples of such agents include anastrozole (Arimidex) and letrozole (Femara). These drugs can be also classified into first-generation (e.g. aminoglutethimide), second-generation (e.g. formestane and fadrazole) and third-generation (e.g. anastrozole, letrozole and exemestane) compounds. Anti-aromatase agents may also be divided into Type I and Type II inhibitors. The Type I inhibitors have a steroidal structure similar to androgens and inactivate the enzyme irreversibly by blocking the substrate-binding site, and are therefore known as aromatase inactivators. Examples of such drugs include formestane and exemestane (Aromasin). Type II inhibitors are non-steroidal and their action is reversible. Examples include anastrozole and letrozole. In one aspect, one or more Ln-5 modulators are provided or combined with one or more of such molecules selected from Formestane, Exemestane, Aminoglutethimide, Anastrozole, and Letrozole.

Prostate cancer is often sensitive to finasteride, an agent that blocks the peripheral conversion of testosterone to 5-hydroxy-testosterone. Ln-5 modulators can be provided or combined with this agent or provided in association with various forms of androgen deprivation therapy (ADT).

In one aspect, one or more Ln-5 modulators are combined with or co-delivered with one or more intracellular signaling inhibitors. Examples of such compounds include tyrosine kinase inhibitors (Gleevec®, imatinib mesylate), modulators of the ras signaling pathway, and regulators of protein trafficking. Other examples include serine/threonine kinase inhibitors, protein-tyrosine phosphatases inhibitors, dual-specificity phosphatases inhibitors, and serine/threonine phosphatases inhibitors.

In another aspect, the invention provides combination compositions and combination delivery methods comprising one or more immune system inhibitors and one or more Ln-5 modulators. Numerous immunosuppressive/immunomodulatory agents are known, examples of which include T lymphocyte homing modulators (e.g., FTY-720—see, e.g., Yangawa et al., J Immunol. 1998 Jun. 1; 160(11):5493-9); calcineurin inhibitors (such as valspodar, PSC 833, and other MDR-1 or p-glycoprotein inhibitors); and TOR-inhibitors (e.g., sirolimus, everolimus, and rapamcyin).

Other features the invention are combination compositions and combination delivery methods comprising one or more Ln-5 modulators and one or more antineoplastic antibiotics. Such antibiotic chemotherapy agents prevent or delay cell replication. There are many differing antitumour antibiotics, but generally they prevent cell division by two ways: (1) binding to DNA making it unable to separate (2) inhibiting ribonucleic acid (RNA), preventing enzyme synthesis. Examples of such agents include Bleomycin (Blenoxane), Dactinomycin (Actinomycin D, Cosmegen), Daunorubicin (Cerubidine), Doxorubicin (Adriamycin, Rubex), Idarubicin (Idamycin), Mitomycin (Mitomycin-C, Mutamycin), Mitoxantrone (Novantrone), Pentostatin (Nipent), Plicamycin (Mithracin, Mithramycin), and combinations thereof.

In other aspects, a Ln-5 modulator or related composition is delivered to a host in association with a thrombosis modulating agent such as a low molecular weight heparin, standard heparin, pentasaccharides, thrombin inhibitory agents (melagatran, ximelagatran, etc.), and/or coagulation factors like Factor VII, Factor VIII, etc.

Chemotherapeutic drugs may lack the ability to adequately penetrating tumors to kill them because these cells may be dead or lack a good blood supply. Anaerobic bacteria, such as Clostridium novyi, can consume the interior of oxygen-poor tumors. Such bacteria die when they come in contact with the tumor's oxygenated sides, meaning they are likely harmless to the rest of the body. The application of such bacteria and one or more Ln-5 modulators represents another feature of the invention. Typically, such methods are practiced in further combination with a chemotherapeutic agent.

As indicated above, various methods effective in the treatment of cancer can be combined with the delivery of an effective amount of one or more Ln-5 modulators to a subject. Particular examples of such techniques are described in further detail here.

In one aspect, the invention provides a combination method that comprises application of radiation or associated administration of radiopharmaceuticals to a patient in combination with one or more Ln-5 modulators. In a related facet, the invention provides a composition comprising an effective combination of one or more Ln-5 modulators and one or more radiopharmaceuticals.

The source of radiation in such methods can be either external or internal to the patient being treated (radiation treatment can, for example, be in the form of external beam radiation therapy (EBRT) or brachytherapy (BT)). Radioactive elements that can be used in practicing such methods and included in such compositions include, e.g., radium, Cesium-137, Iridium-192, Americium-241, Gold-198, Cobalt-57, Copper-67, Technetium-99, Iodide-123, Iodide-131, and Indium-111. Additionally useful radionuclides that can be incorporated in radiopharmaceuticals and used in such methods are discussed elsewhere herein (e.g., in the context of Ln-5 modulator conjugates). In a particular aspect, such methods and compositions also further optionally include one or more radiation protectors. These drugs are designed to protect normal cells from radiation. One example is the intravenous drug amifostine (Ethyol). In another particular aspect, intensity-modulated radiation therapy (IMRT) is applied in combination with the delivery of one or more Ln-5 modulators. IMRT delivers radiation therapy that is targeted to tumor shape, minimizing damage to healthy tissue. More particularly, IMRT allows radiation beams to be divided up and delivered in different intensities and directions to match the tumor's shape. Another similar technique, three-dimensional conformal radiation therapy, has been demonstrated to be effective in some situations and can be combined with Ln-5 modulator therapeutic and prophylactic regimens. Pulsed delivery (gating) of radiation also or alternatively can be used in such methods, reducing the field of radiation by compensating for natural breathing patterns.

In a further facet, Ln-5 modulators are provided in association with the application of a radiogenic therapy. One example of such a therapy is the localized production of cytotoxic agents by radiation stimulation/activation of a prodrug or gene associated with a radiation-inducible promoter (such a gene may code for a cytotoxic protein, an enzyme that activates a co-delivered prodrug, etc.). Another example, is targeted auger-emitting radiolabeled molecules. These therapies can control cancer by delivering targeted radiation to specific receptor bearing cells. Auger electrons are emitted by radioactive isotopes (Iodine-125 or Indium-111). The electrons have very short ranges and therefore have the potential to be delivered to specific sets of target cells, sparing healthy cells. In another exemplary method, a nucleic acid comprising a radiation-induced gene sequence that codes for a protein that can be targeted by a cytotoxic agent is delivered in association with one or more Ln-5 modulators. Radiation is applied to produce the protein and the cytotoxic agent delivered so as to provide a targeted therapy.

In further aspects, Ln-5 modulators are delivered in connection with application of photodynamic therapy. In general, such therapies involve the delivery of a photosensitizing agent that makes cells more sensitive to light and, by doing so, causes cancer cells to be destroyed when a laser light is directed on a cancerous area. Thus, various prophylactic and therapeutic regimens of the invention also or alternatively can be combined with anti-cancer directed photodynamic therapy (e.g., anti-cancer laser therapy—which optionally can be practiced with the use of photosensitizing agent, see, e.g., Zhang et al., J Control Release. 2003 Dec. 5; 93(2):141-50)). Rhodium compounds, for example, can damage DNA in living cells in a manner similar to platinum classic chemotherapy drugs, while remaining benign until irradiated with light.

Lasers also can be used in the performance of precise anti-cancer surgeries (e.g., where labeled Ln-5 modulators have identified cancerous tissues and/or precancerous growths). Other forms of surgery also or alternatively can be applied in connection with the delivery of one or more Ln-5 modulators. Anti-cancer surgical techniques (e.g., colectomy, proctocolectomy, polypectomy, prostatectomy, segmental resection, lobectomy, pneumonectomy, lumpectomy, mastectomy, etc.) are well known in the art and accordingly are not discussed in detail here (see, e.g., CANCER SURGERY, Harvey and Beatie (W.B. Saunders Company 1996); ADVANCED ONCOLOGIC SURGERY, Roh et al. Eds. (Mosby-Year Books, 1^{st} Ed. 1994); CANCER SURGERY, McKenna et al. Eds. (Lippincott Williams & Wilkins 1994); The M. D. ANDERSON SURGICAL ONCOLOGY HANDBOOK, Feig et al. (Lippincott Williams & Wilkins; 3rd Ed. 2002); and SURGICAL ONCOLOGY: CONTEMPORARY PRINCIPLES AND PRACTICE, Bland et al. (McGraw-Hill Professional; 1^{st} Ed. 2001). In a particular aspect, Ln-5 modulator therapy and/or labeled Ln-5 modulator diagnostic techniques is/are combined with anti-cancer cryosurgery. In another aspect, organs (such as the ovaries or testicles) that make the hormones may be removed in connection with Ln-5 modulator anti-cancer therapy.

In a further aspect, Ln-5 modulator therapy is combined with the application of a bone marrow transplant and/or anti-cancer stem cell therapy. Stem cell transplantation (SCT), for example, may advantageously used in cancer treatment. The SCT may be autologous (the person's own cells that were saved earlier), allogeneic (cells donated by another person), or syngeneic (cells donated by an identical twin). SCT methods and related principles are known in the art (see, e.g., Georges et al., Int J Hematol. 2003 January; 77(1):3-14; Tabbara et al., Anticancer Res. 2003 November-December; 23(6D):5055-67; Bhatia et al., Expert Opin Biol Ther. 2001 January; 1(1):3-15; Huugen et al., Neth J Med. 2002 May; 60(4):162-9; Margolin et al., J Urol. 2003 April; 169(4):1229-33; and U.S. Pat. No. 6,143,292). Bone marrow transplant is an even more well known method used in treatment of certain cancers (see, e.g., Thomas, Ann N Y Acad Sci. 1995 Dec. 29; 770:34-41; Kolb and Holler, Stem Cells. 1997; 15 Suppl 1:151-8; Thomas, Semin Hematol. 1999 October; 36(4 Suppl 7):95-103).

Ln-5 modulators also can be delivered in association with application of other therapeutic methods such as anti-cancer sound-wave and shock-wave therapies (see, e.g., Kambe et al., Hum Cell. 1997 March; 10(1):87-94); anti-cancer thermotherapy (see, e.g., U.S. Pat. No. 6,690,976), and/or anti-cancer neutraceutical therapy (see, e.g., Roudebush et al., Vet Clin North Am Small Anim Pract. 2004 January; 34(1):249-69, viii and Rafi, Nutrition. 2004 January; 20(1):78-82). Other methods include diet therapies (e.g., fasting therapy (which may be aided by anti-obesity agents or anti-appetite agents) or adoption of a high potassium, low sodium (saltless) diet, with no fats or oils, and high in fresh raw fruits and vegetables—see, e.g., A Cancer Therapy: Results of Fifty Cases, Max Gerson, Gerson Inst; 6th edition). Another technique that may be combined with Ln-5 modulator anti-cancer therapy is inuslin potentiation therapy, wherein low-dose insulin is given in conjunction with low-dose chemotherapy and Ln-5 modulator anti-cancer therapy.

Ln-5 modulator anti-cancer methods also can be applied in conjunction with various adjunct therapies designed to ameliorate cancer-associated and cancer treatment-associated conditions, such as treatments for depression, treatments for pain (e.g., by delivery of morphine or a morphine derivative), treatment for incontinence, treatment for impotence, etc.

The inventive methods described herein also or alternatively can be practiced in connection with the delivery of one or more agents that promote access of an Ln-5 modulator, related compound, or combination thereof to the interior of a tumor. Thus, for example, such methods can be performed in association with the delivery of a relaxin, which is capable of relaxing a tumor (see, e.g., U.S. Pat. No. 6,719,977). As another example of such a technique, an Ln-5 modulator can be bonded to a cell penetrating peptide (CPP). Cell penetrating peptides and related peptides (such as engineered cell penetrating antibodies) are described in, e.g., Zhao et al., J Immunol Methods. 2001 Aug. 1; 254(1-2):137-45; Hong et al., Cancer Res. 2000 Dec. 1; 60(23):6551-6; Lindgren et al., Biochem J. 2004 Jan. 1; 377(Pt 1):69-76; Buerger et al., J Cancer Res Clin Oncol. 2003 December; 129(12):669-75; Pooga et al., FASEB J. 1998 January; 12(1):67-77; and Tseng et al., Mol Pharmacol. 2002 October; 62(4):864-72. Intratumoral administration of L5G2D3BPs or vectors comprising L5G2D3BP-encoding or related molecule-encoding nucleic acid sequences also or alternatively can be used to facilitate therapeutic regimen aspects of the invention.

Additional target Ln-5-binding molecules and molecules that are involved with Ln-5-influenced aspects of cancer progression and, accordingly, are advantageous targets for secondary molecules in the context of combination compositions and/or combination delivery methods include α6β1 integrin, α3β1 integrin, α2β1 integrin, α6β1 integrin, laminin-6, laminin-7, EGF-R, type VII collagen, fibulin-1, fibulin-2, Rho GTPases, BP180, syndecan-4, nidogen-1, phosphorylated hsp-27, p300, a cytokeratin, and other matrix metalloproteinases (e.g., MMP-1, MMP-2, MMP-9, MMP-10 and MMP-14 (which also known as Membrane-type matrix metalloproteinase 1 (MT1)), tissue inhibitor of matrix metalloproteinase-1 (TIMP-1) and TIMP-2, E-cadherin, bone morphogenic protein-1 (BMP-1), and the 67 kDa laminin receptor. Thus, in one aspect, the invention provides a method of reducing a cancer progression aspect (e.g., cancer cell migration) in a human patient in need thereof comprising delivering a Ln-5 modulator and an antibody specific for one or more of these non-similar molecules to the patient in amounts and under conditions such that cancer progression is detectably reduced in the patient. Additional types of such molecules are discussed elsewhere herein and/or are known in the art.

In other aspects, the Ln-5-associated antibody and/or Ln-5 binding protein (or other Ln-5 modulator) is administered in association with a thrombosis modulating agent such as a low molecular weight heparin, standard heparin, pentasaccharides, thrombin inhibitory agents (melagatran, ximelagatran, etc.), and/or coagulation factors like Factor VII, Factor VIII, etc. In another aspect, the anti-cancer agent is an anti-angiogenic agent, such as an anti-VEGF agent (e.g., an anti-VEGF agent such as Avastatin), angiostatin, canstatin, Combrestatin, endostatin, NM-3, Thrombospondin, Tumstatin, 2-methoxyestradiol, Vitaxin, etc. or other angiogenesis modulator such as interferon alpha, inhibitors of hypoxia-inducible factor 1, IL-12, Marimastat, Neovastat, Angiozyme, Thalidomide, Squalamine, etc. In still another aspect the anti-cancer agent is an estrogen receptor modulator (SERM) such as tamoxifen, idoxifene, fulvestrant, droloxifene, toremifene, raloxifene, diethylstilbestrol, ethinyl estradiol/Estinyl, etc. The anti-cancer agent also can be an aromatase inhibitor like anastrazole/Arimidex, aminoglutethimide/Cytraden, Exemestane, etc.; an antiandrogene like Flutaminde/Eulexin; a progestin, such as hydroxyprogesterone caproate, Medroxyprogesterone/Provera, Megestrol acepate/Megace, etc.; an adrenocorticosteroid like hydrocortisone, prednisone, etc.; a luteinising hormone-releasing hormone (LHRH) analogue like buserelin, goserelin, etc.; a hormone inhibitor such as octreotide/Sandostatin, etc.; a growth suppressive agent, such as antimetabolites like Cepecitabine/Xeloda, cytarabine/Ara-C, Cladribine/Leustatin, Fludaraine/Fludara, fluorouracil/5-FU, gemcitabine/Gemzar, mercaptopurine/6-MP, methotrexate/MTX, thioguanine/6-TG, Allopurinol/Zyloprim, etc.; an acylating agent such as Busulfan, Cyclophosphamide, mechlorethamaine, Melphalan, thiotepa, semustine, carboplatin, cisplatin, procarbazine, dacarbazine, Althretamine, Lomustine, Carmustine, Chlorambucil, etc.; a topoisomerase inhibitor like Camptothecins as Topotecan, Irinotecan; like Podophyllotoxins as Etoposide/VP16, Teniposide/VP26, etc.; an inhibitor of microtuble and/or spindle formation, such as Vincristine, Vinblastine, Vinorelbine, Taxanes as Paxlitaxel, Docetaxel, combrestatin, Epothilone B, etc.

Anti-cancer agents also include antibiotics like bleomycin, Dactinomycin/Actinomycin D, anthracyclins as Daunorubicin/Cerubidine and Doxorubicin, idarubicin, mitomycins, plicamycin, etc.; telomerase inhibitors; differentiation inducing agents; retinoic acid analogues like all trans retinoic acid, 13-cis retinoic acid, etc.; vitamin D analogues like seocalcitol, etc.; inhibitors of receptor tyrosine kinases; inhibitors of ErbB-1/EGFR like iressa, Erbitux, etc.; inhibitors of ErbB-2/Her2 like Herceptin, etc.; inhibitors of c-kit like Gleevec; inhibitors of VEGF receptors like ZD6474, SU6668, etc.; Inhibitors of ErbB3, ErbB4, IGF-R, insulin receptor, PDGFRa, PDGFRbeta, Flk2, Flt4, FGFR1, FGFR2, FGFR3, FGFR4, TRKA, TRKC, c-met, Ron, Sea, Tie, Tie2, Eph, Ret, Ros, Alk, LTK, PTK7, etc.; cancer related enzyme inhibitors such as metalloproteinase inhibitors like marimastat, Neovastat, etc. Other anti-cancer agents include cathepsin B, modulators of cathepsin D dehydrogenase activity, glutathione-5-transferase like glutacylcysteine synthetase, and lactate dehydrogenase

In other aspects, virus delivery using a modified virus (usually replication deficient) can be used as a delivery vehicle. For example, adenovirus, measles virus and other vira can be used to deliver genes encoding, e.g., tumor suppressor genes such as human recombinant wild-type p53/SCH58500, Apc, PTEN etc., or genes encoding a death receptor ligand like TRAIL, TNF etc. The viral delivery vehicle can also be used to deliver antisense nucleic acids to oncogenes, mutated or deregulated genes, siRNA to mutated or deregulated genes such as Ras, Carcinoembryonic antigen (CEA). Also, delivery of genes encoding interleukins resulting in the attraction of immunoresponse like TNF, IL-2 etc., can be made using such vehicles

In additional aspects, an anti-cancer agent can be a HSP90 inhibitor like 17-allyl amino geld-anamycin; antibodies directed against a tumor antigen such as PSA, CA125, KSA, etc.; integrins like integrin beta1; and inhibitors of VCAM. Other anti-cancer agents include mycophenolate mofetil, mycophenolic acid, asparaginase, calcineurin-inhibitors, TOR-inhibitors, etc., and inhibitors of “lymphocyte homing” mechanisms such as FTY720, etc., and agents with effects on cell signaling such as adhesion molecule inhibitors (e.g., anti-LFA, etc.).

Methods of promoting the treatment and/or prevention of these and other diseases and disorders by administering an Ln-5 modulator under conditions such that one or more functions of Ln-5 and/or one or more functions of one or more biological molecules associated with Ln-5 (normally and/or in the relevant disease state(s) in the applicable patient or host) are at least partially (e.g., at least substantially, or essentially entirely) abrogated are another important feature of this invention.

The invention further provides method of promoting the sale and/or use of a compound according to any of the preceding aspects, or otherwise described herein, comprising distributing information related to the use of the compound in the prevention or treatment of any condition or combination of conditions recited in any of the foregoing aspects or described elsewhere herein.

Pharmacological Methods

Animal and cell models of IBD or polycystic kidney disease (PKD) also can be useful in assessing the effectiveness of a particular Ln-5-associated antibody, Ln-5 binding protein, or other Ln-5 modulator, or in various combinations thereof, in promoting, enhancing, and/or inducing the desired response(s) discussed herein.

For example, colonic inflammation can be induced in mice, rats, or another mammalian models by known techniques, and a macroscopic damage score reflective of the amount of ulceration and/or inflammation in the colon of the mammals can be determined by such techniques in control and test groups as a means of assessing the ability of an agent to reduce IBD. Other similarly known animal models measure colonic strength and/or stiffness of colon tissue as a means of evaluating a particular therapeutic or prophylactic regimen. Specific models include the trinitrobenzene sulphonic acid (“TNBS”) model for IBD. The TNBS model is one of the standard IBD models used in IBD discovery research and has been extensively evaluated in rodents. See, for example, C. O. Elson et al. (1995), Experimental Models of Inflammatory Bowel Disease, Gastroenterology, 109: 1344-1367 and references cited therein. Another specific model is the Dextran Sulfate (“DSS”) model. Similar to the TNBS model, DSS-induced colitis is widely used as a screening tool for IBD therapeutics. Another method of evaluating IBD is the use of chemiluminescence assays of mucosal reactive oxygen metabolites. Eosinophil-associated neurotoxins also can be a marker for IBD (see, e.g., U.S. Pat. No. 5,928,883). The TNO Pharma model of Inflammatory Bowel Disease has a good reproducibility and low mortality compared to described models. This has been achieved by sensitization of the animals prior to the rectal administration of TNBS. The model has successfully been validated with budesonide and sulfasalazine, which are two of the most used pharmaceutical compounds for the treatment of IBD. Anti-Saccharomyces cerevisiae antibodies (ASCA) can also be useful in the assessment of IBD, as can be anti-neutrophil cytoplasmic antibodies (ANCA) that demonstrate perinuclear (P-ANCA) or atypical pattern. Commercially available systems, such as the Prometheus system, IBD FIRST STEP and IBD DIAGNOSTIC SYSTEM (Prometheus Laboratories, Inc.—San Diego, Calif.) also can be used in assessing IBD.

Thus, selection of therapeutic Ln-5 modulators for treatment of IBD, such as antibodies binding Ln-5 useful in the context of IBD treatment, can be selected by immunohistochemistry (IHC) detecting staining of a IBD site/diseased site without or with reduced staining of normal basal lamina of the skin or vessels. Also, IBD can be induced in animals, preferably in mice or rats, be adding Dextran Sodium Sulfat (DSS) to their drinking water. The Ln-5-associated antibody, Ln-5 binding protein, and/or other Ln-5 modulator composition is administered to the animals preferably by, but not limited to, injection. Clinical signs of IBD and biochemical markers are recorded to allow for discrimination between effects of the compositional matter and vehicle treatment.

In another example, IBD is induced in animals, preferably in mice or rats, by rectal instillation of 2,4,6-Trinitrobenzenesulfonic acid (TNBS) solubilized in ethanol. The Ln-5 modulator (e.g., a Ln-5-associated antibody and/or Ln-5 binding protein) is administered to the animals preferably by, but not limited to, injection. Clinical signs of IBD and biochemical markers are recorded to allow for discrimination between effects of the compositional matter and vehicle treatment.

In an illustrative aspect, the effect of an Ln-5 modulator is evaluated in a model where IBD is induced by an adoptive transfer. Adoptive transfer of CD4+ CD25− T-cells to immunodeficient mice (SCID or RAG-KO) induces a colitis that is clinically manifest by 2½ weeks after transfer. The colitis is characterized clinically by weight loss, diarrhoea and blood in faeces. The macroscopic appearance of the colon is dominated by severe hypertrophy of gut wall which is can also be observed by histology. Inflammation follows hypertrophy 3-5 weeks after transfer, while ulcerations are only seen in later stages. Dysplasia or metaplasia is not normally encountered. The pathology is mainly confined to the large intestine (including the cecum) with no histological signs of gastritis, pancreatitis or sialitis. For testing the efficacy of a functional Ln-5 modulator with a physiological effect on IBD, CD4+ CD25− T-cells are purified from spleens of syngeneic donor mice by MACS (magnetic activated cell sorting). To each mouse, 3×10⁵ CD4+ CD25− T-cells are injected i.p. in a volume of 200 μl RPMI 1640 with glutamax. Mice are weighed twice weekly and any signs of illness are noted. The Ln-5 modulator is administered via a route that is compatible for optimal bioavailability, for example, i.v., p.o., i.p., s.c., or other suitable route. If needed, the Ln-5 modulator can be administered repeatedly during an appropriate period of time. For example, the frequency of administration can vary from one or more times daily to every second day, third day, weekly, or monthly, and interval treatment can be used if appropriate. In one embodiment, for an antibody Ln-5 modulator, a dose of 20 mg/kg dosed i.p. in PBS could be administered twice weekly. To evaluate prophylactic efficacy or prevention or delay in disease or disease symptoms, the Ln-5 modulator can be administered prior to adoptive transfer. Alternatively, to measure Ln-5 modulator treatment efficacy on disease progression, disease development, or the severity of the disease, the Ln-5 modulator dosing regimen can be initiated 1 to 3 weeks after the adoptive transfer. Animals are then treated for 2½-6 weeks before sacrifice. Prior to sacrifice, 100 mg/kg BrdU is injected i.p. in 200 μl NaCl physiological solution. At necropsy, the entire gastrointestinal tract is removed for dissection. The weight, length and width of the gastrointestinal tract is determined before the tissue is prepared for immunohistochemisty. Relevant physiological effects include a significant normalization of animal weight and/or reduced incidence of blood in faeces and/or reversion to normal gut weight, length and/or thickness and/or significant reduction in proliferation (BrdU staining) and/or significant normalisation of mucin production (determined by PAS or alcian blue or alcian blue HID staining) and/or preservation of goblet cells. Reversion or reduction of the inflammation in the IBD state is also a physiological effect determining efficacious LN-5 modulators.

In another illustrative aspect, the effect of an Ln-5 modulator is evaluated in a mouse model where colitis is induced by DSS. Briefly, eight week old male wild-type C57Bl/6 mice are treated with or without DSS, appended to their drinking water at a concentration of 1.5%, for 8 days. For example, in initial pilot experiments, a concentration of DSS was titrated from 1-5% and given to wild-type mice; and a dose that resulted in disease after 8 days was chosen for subsequent experiments. Titration of the DSS dose is important. Even a change of location can affect the results, since the kinetics of disease induction depends on the type and amount of microbes in the specific location. After 6-8 days, mice begin loosing weight. Mice are then weighed twice weekly and any signs of illness are noted. The efficacy of a functional Ln-5 modulator with a physiological effect on IBD can be determined by administration of the compound by a route that is compatible for optimal bioavailability and can be either i.v., p.o., i.p., s.c. or other if suitable. If needed, the Ln-5 modulator can be administered repeatedly during an appropriate period of time. For example, the frequency of administration can vary from one or more times daily to every second day, third day, weekly, or monthly, and interval treatment can be used if appropriate. In one embodiment, for an antibody Ln-5 modulator, a dose of 20 mg/kg dosed i.p. in PBS could be administered twice weekly. The first dose could be administered the day before addition of DSS to the drinking water, and administrations continued during 8 to 15 days. Before sacrifice, 100 mg/kg BrdU is injected i.p. in 200 μl NaCl physiological solution. At necropsy the entire gastrointestinal tract is removed for dissection. The weight, length and width of the gastrointestinal tract is determined before the tissue is prepared for immunohistochemisty. Physiological effects indicative of treatment include a significant normalization of animal weight and/or reduced incidence of blood in faeces and/or reversion to normal gut weight, length and/or thickness and/or significant reduction in proliferation (BrdU staining) and/or significant normalisation of mucin production (determined by PAS or alcian blue or alcian blue HID stainings) and/or preservation of goblet cells. Reversion or reduction of the inflammation in the IBD state is also a physiologial effect determining efficacious Ln-5 modulators. Ln-5 expression in the DSS model has been described by Pirila et al., Dig Diseases Sci. 2003; 48:93-98.

The efficacy of a functional Ln-5 modulator with a prophylactic effect to reduce or abrogate the increased cancer risk in IBD patients can be detected in mice by evaluating the efficacy against nascent adenomatous polyps in the mouse models of multiple intestinal neoplasia (min), the Apcmin model. Inbread B6-Apcmin mice can be obtained from, e.g., The Jackson Laboratory and screened for the Min/+genotype using PCR assay. Mice are divided into groups normalizing distribution of males and females, avoiding the clustering of mice from a single litter. Mice are monitored for health every 3 days and weighed weekly. Treatment is initiated at age 28 days (at the time of weaning) and continued for several weeks, optionally until day 100 of age. The compound is administered by a route that is compatible for optimal bioavailability and can be either i.v., p.o., i.p., s.c. or other suitable route. If needed, the Ln-5 modulator can be administered repeatedly during an appropriate period of time. For example, the frequency of administration can vary from one or more times daily to every second day, third day, weekly, or monthly, and interval treatment can be used if appropriate. In an exemplary embodiment, the Ln-5 modulator is an antibody, and administered i.p. in PBS at a dose of 20 mg/kg twice weekly. At the end of the treatment period, when the mice are 90-115 days of age, the animals are sacrificed. At necropsy the entire gastrointestinal tract is removed for dissection. The small intestine is divided into three segments of approximately equal length, and the colon is left intact. All four intestinal segments are opened longitudinally and washed extensively with PBS to remove intestinal contents. Tissues are fixed (60% methanol, 30% chloroform, 10% acetic acid) and rinsed (70% ethanol) before tumor enumeration is performed. Tumor size (area mm²) is measured using image analysis of photographed intestinal tissues mounted on a calibrated stage micrometer. The smallest tumors scored are 0.2 mm in diameter for counting. Statistically significant (P<0.05) change in tumor number and/or tumor size are obtained for an efficacious LN-5 modulator.

As mentioned above, animal models of ADPKD are known and have been described by, e.g., Thomson et al. (2003 November; 285(5):F870-80). Further, the effect on renal cystic disease development and progression of an LN-5 modulating agent can be established in animal models as described in, e.g., Gattone et al. (2003) Nature Med. 9 (10):1323-1326 or in Qian et al. (2004) J. Biol. Chem (Epub ahead of print). Based on the administration of an effective amount of an LN-5 modulating agent to mice or rats (depending on model), and following the development or progression of disease over 3 to 30 weeks, the functionality of an Ln-5 modulator can be established. Relevant indicators of treatment efficacy include a reduction in renal cyst number or size, renal fibrosis volume in combination with or indicated by reduction of renal cAMP (pmol/mg wet tissue), and or plasma BUN (blood urea nitrogen). In a particular embodiment, two models of PKD are tested. One model of PKD is in rat, where a splice mutation leads to a frameshift of the gene Pkhd1 which is the rat ortholog of PKHD1, thereby causing the disease. Another PKD model is pcy mice. Animals from both models have a defect in urine concentration and a significant higher renal cAMP. In an exemplary assay, animals are monitored for health twice weekly and weighed weekly. Urine is collected during the experiment for measuring osmolarity. The administration of an Ln-5 modulator to PCK rats is initiated by 3 weeks of age, and continues to the age of 10 weeks. For pcy mice treatment is initiated by week 4 of age, and continues to age 30. The Ln-5 modulator is administered by a route that is compatible for optimal bioavailability and can be either i.v., p.o., i.p., s.c., or other suitable route. If needed, the Ln-5 modulator can be administered repeatedly during an appropriate period of time. For example, the frequency of administration can vary from one or more times daily to every second day, third day, weekly, or monthly, and interval treatment can be used if appropriate. In the case where the Ln-5 modulator is an antibody, and exemplary treatment regimen would employ a dose of 20 mg/kg dosed i.p. in PBS and administered twice weekly. At the end of treatment, animals are sacrificed. Two hours before sacrifice, 100 mg/kg BrdU is injected i.p. in 200 μl NaCl physiological solution. At necropsy the blood is collected by cardiac puncture for analysis and the kidneys are removed for dissection. The weight of the kidneys and the visual appearance is noted. The left kidney is fixed in 10% formaldehyde in phosphate buffer (pH 7.4) embedded in paraffin for histological studies. The right kidney is immediately frozen in liquid nitrogen for determination of cAMP. The histological studies use H&E staining to measure cyst volume and picrosirius red collagen stain to measure fibrosis both combined with image analysis. Apoptotic index is measured by TUNEL assay and mitotic index by BrdU staining. The blood is analysed for blood urea nitrogen and serum creatinine. The right kidneys are grounded in a homogeniser in ten volumes of cold 5% trichloroacetic acid. After centrifugation 600×g 10 minutes the supernatants are extracted with three volumes of water-saturated ether. After drying the extracts are reconstituted in a buffer for analysis in an enzyme immunoassay using kit (Sigma-Aldrich). Physiological effects of an efficacious LN-5 modulator can be seen as markedly reducing the renal accumulation of cAMP measured as pmol/mg wet kidney or as pmol/mg protein. Significant (p<0.05) reduction in renal cysts and/or fibrosis and/or kidney weight and/or renal proliferation and/or renal apoptotic index are exemplary indicators of a useful Ln-5 modulator, as are normalization of urine osmolarity and/or blood urea nitrogen and/or serum creatinine.

These methods provide techniques for identifying/screening for Ln-5 modulators, such as Ln-5-associated antibodies and/or Ln-5 binding proteins, with superior anti-IBD and/or anti-ADPKD properties.

Accordingly, the invention further provides a method of screening candidate compositions for biological activity associated with the prevention and/or treatment of IBD in a mammal comprising providing a composition comprising an Ln-5-associated antibody, an Ln-5 binding protein, or both, in an amount expected to result in a response in a model of IBD that is predictive of a correlated therapeutic or prophylactic effect in a mammal, subjecting the composition to analysis by the model, and assessing the therapeutic and/or prophylactic potential the composition by assessing the effect of the composition in the model.

In an additional aspect, the invention provides a method of screening candidate compositions for biological activity associated with reducing the spread and/or growth of renal cysts in a mammal comprising providing a composition comprising an Ln-5-associated antibody, an Ln-5 binding protein, or both, in an amount expected to result in a response in a model of ADPKD that is predictive of a correlated therapeutic or prophylactic effect in a mammal, subjecting the composition to analysis by the model, and assessing the therapeutic and/or prophylactic potential the composition by assessing the effect of the composition in the model.

In a yet further aspect, the invention provides a method of screening candidate compositions for biological activity associated with treating or preventing irritable bowel syndrome comprising providing a composition comprising an Ln-5-associated antibody, an Ln-5 binding protein, or both, in an amount expected to result in a response in a model of irritable bowel syndrome that is predictive of a correlated therapeutic or prophylactic effect in a mammal, subjecting the composition to analysis by the model, and assessing the therapeutic and/or prophylactic potential the composition by assessing the effect of the composition in the model.

The invention additionally provides a method of screening candidate synergistic combinations of anti-cancer agents comprising providing a composition comprising a combination of an Ln-5-associated antibody, an Ln-5 binding protein, or both, and at least one secondary anti-cancer agent in amounts expected to result in a response in a model of that is predictive of a correlated therapeutic or prophylactic effect in a mammalian host, obtaining a predicted cumulative effect for the combination, subjecting the composition to analysis by the model, and assessing whether the combination results in a result that is substantially greater than the predicted cumulative effect for the combination so as to identify a synergistic combination of anti-cancer agents.

Pharmaceutical Compositions

In still another aspect, the invention provides a pharmaceutical product comprising (a) a composition according to any of the foregoing aspects or elsewhere described herein, (b) a pharmaceutically acceptable carrier, vehicle, excipient, diluent, preservative, stabilizer, binder, flavoring agent, an antioxidant, a colorant, adjuvant, disintegrating agent, solvent, a solubilizer, a suspending agent, a isotonizing/isotonic agent, a buffer, a soothing agent, or combination of any thereof (including any multiples thereof—e.g., two diluents), and, optionally, (c) a notice associated with said container in form prescribed by a governmental agency regulating the manufacture, use, or sale of pharmaceuticals, which notice is reflective of approval by said agency of said pharmaceutical product for human or veterinary administration to treat at least one condition recited in any of the foregoing aspects or other condition or disease described herein.

The compounds of the present invention may be administered alone, in combination with other active agents as described herein, and/or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral, parenteral, and intraperitoneal routes being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art. Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention. Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. Examples are an acid addition salt of a compound having the utility of a free base and a base addition salt of a compound having the utility of a free acid. The term “pharmaceutically acceptable salts” refers to non-toxic salts of the compounds of this invention which are generally prepared by reacting the free base with a suitable organic or inorganic acid or by reacting the acid with a suitable organic or inorganic base. When a compound according to the present invention contains a free base such salts are prepared in a conventional manner by treating a solution or suspension of the compound with a chemical equivalent of a pharmaceutically acceptable acid. When a compound according to the present invention contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds of the present invention and these form a further aspect of the present invention.

For parenteral administration, solutions of the novel compounds of the formula (I) in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the novel compounds of the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil. Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin. Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid. Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present. The pharmaceutical compositions of the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents. Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the present invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example. For topical use, creams, ointments, jellies, solutions of suspensions, etc., containing the compounds of the present invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds of the present invention may form solvates with water or common organic solvents. Such solvates are also encompassed within the scope of the present invention.

Thus, in a further embodiment, there is provided a pharmaceutical composition comprising a compound according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non aqueous liquid suspension or solution.

The Ln-5 modulator compositions described herein can be, in and of themselves (i.e., apart from the inventive methods described here) important features of the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Unless explicitly indicated or clearly contradicted by context, approximate values described herein (e.g., “about 10”) may be replaced by corresponding exact values, and visa versa.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor(s) for carrying out the invention. Variations of those preferred embodiments may become apparent to those of ordinary skill in the art upon reading the foregoing description. The inventor(s) expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### IHC Immunohistochemical Detection of Laminin-5 γ2 Expression in IBD

This example describes the detection of Ln-5 γ2 chain expression in an animal model of IBD.

Animal model. CD4+ CD25− T-cells were purified from spleens of syngeneic donor mice by MACS (magnetic activated cell sorting). To each mouse, 3×10⁵ CD4+ CD25− T-cells were injected i.p. in a volume of 200 μl RPMI 1640 with glutamax. Mice were weighed twice weekly and any signs of illness were noted.

Immunohistochemistry: The mice were injected with Bromodeoxyuridne (BrdU, 100 mg/kg) 2 h before sacrifice. Colon was dissected, fixed in paraformaldehyde and embedded in paraffin. Colon sections (4 μm) from control SCID mice and IBD mice were de-paraffinized in xylene and rehydrated. Microwave oven-mediated antigen retrieval was performed in Tris/EDTA buffer, pH=9, and endogeneous peroxidase activity was blocked with 0.5% hydrogen peroxide in Tris buffered saline (TBS) for 20 minutes at room temperature (RT). Endogeneous biotin was blocked using the biotin blocking system from DakoCytomation (Glostrup, Denmark).

For Ln-5 γ2 immunostaining, slides were incubated in TBS containing 4% casein (tryptone casein peptone, Ameresco) for 1 hour at RT to reduced non-specific background, and then stained with 1 μg/ml rabbit polyclonal anti-human Ln-5 γ2 antibody Pab28 (obtained from Sirpa Salo, Oulo, Finland) diluted in 4% casein/TBS at 4° C. overnight. The secondary antibody biotinylated donkey anti-rabbit (1:3000, Jackson) was applied for 1 hour at RT. Signal amplification was performed by incubation with horseradish peroxidase-conjugated streptavidin and biotinylated Tyramide according to the manufacturer (NEN, Perkin Elmer Life Science, Boston, Mass.). Slides were developed using diaminobenzidine and nuclei were counterstained with hematoxylin.

For BrdU staining, slides were incubated in TBS containing 10% donkey serum and 0.01% Triton-x-100 for 1 hour at RT to reduced non-specific background, and then stained with mouse monoclonal anti-BrdU antibody M744 (1:50, DakoCytomation) diluted in TBS with 0.01% Triton-x-100, 7% donkey serum and 3% rat serum at 4° C. overnight. The secondary antibody, biotinylated donkey anti-mouse (1:3000, Jackson), was applied for 1 hour at RT. Signal amplification was performed by incubation with Vectastain ABComplex according to the manufacturer (Vector). Slides were developed using diaminobenzidine-nickel and nuclei were counterstained with hematoxylin.

As shown in FIGS. 1 and 2, Ln-5 γ2-immunopositive cells in IBD inflicted tissue were observed in some of the crypt cells or parts of the crypt. The comparison to proliferative index by BrdU staining on an adjacent tissue section (FIGS. 2A versus 2B) revealed the Ln-5 γ2-immunopositive cells as non-proliferative. The Ln-5 γ2-immunopositive cells were primarily found to be non-mucin producing (no vesicles were seen in the cells (FIGS. 1A versus 1B)). Therefore, it could be that these cells are more exposed to chemical and/or pathogen injuries.

### Example 2

### Western Blot Analysis of siRNA Against Gamma2 Chain

This example shows that anti-Ln-5 γ2 siRNA reduces the expression of the Ln-5 γ2 chain in vitro.

SW480 cells obtained from ATCC were grown in DME:F12 supplemented with 10% foetal calf serum and routinely checked for being mycoplasma free. For transfection, the cells were seeded at 3×10⁵ cells/2 ml in 6-well plates. Transfection was carried out after cells had attached at least overnight using Lipofectamin 2000 (Life Technologies). Liposomes were formed in medium without serum by mixing 10 μl lipofectamine 2000 and 3 μg plasmid DNA or 15 μl 20 pmol/μl oligonucleotide in a total volume of 250 μl. The following oligonucleotides were used: #7 (SEQ ID NO:7; control) and #6 (SEQ ID NO:6; control), and siRNA sequences #2 (SEQ ID NO:2), and #5 (SEQ ID NO:5), as well as an anti-sense sequence corresponding to the entire Ln-5 γ2 chain; #14. To prepare the sequence encoding the antisense construct to the Ln-5 gamma2 chain, the following sequence was placed into the expression vector pcDNA3.1+(Invitrogen), ccg atc aaa gat aca ctg cct gga ctt ccc att gca atc aca gac ttc cct cct gga ggt ggc ccg ggc tgc ggg cag gag gag cga gaa gca gag gca gca (SEQ ID NO:8).

After liposome formation, the mixture was added to cells and incubated 5 hours in cell incubator before medium was changed to complete medium with serum. After 24 hours, cells were analysed for the effect on laminin-5 γ2 expression.

Media was collected and centrifuged. Attached cells were scraped with a rubber policeman in icecold PBS. The cell pellet from media was pooled with the scraped cells and spun again (1200 rpm 3 min). The supernatant was discharged and cells lysed on ice with 20 μl lysis buffer (50 mM Hepes, pH 8.0, 150 mM NaCl, 1% v/v Triton X100, 2 mM EDTA, 10% v/v glycerol). Cells were spun 13,000×g for 5 min and loading buffer added to a final concentration of 1×(6.57 μl of a 4× loading buffer). Samples were sonicated on ice before spinning as before. The samples were heated to 90° C. for 10 min before loading to individual lanes on a pre-cast gel (NuPAGE 4-12% BisTris) and electrophoresis for 1 hour at 180 V. For size determination and blotting efficiency, 5 μl rainbow marker and 2 μl biotin labelled marker were loaded in individual lanes. The proteins were electroblotted onto nitrocellulose membrane 90 V 3 hours in wet blotting, and detected by western blot.

In order to detect an internal control for protein loading, the membrane was cut into two parts using the rainbow marker to find the approximate size of 50-60 kDa. Two parallel immunodetections were carried out using the membrane with high molecular weight proteins for laminin-5 γ2 detection and the membrane with low molecular weight for actin detection (internal protein control for loading). The membranes were blocked for 1 hour at 25° C. (blocking buffer: 5% non-fat dry milk, 0.1% tween-20 in PBS). After blocking, membranes were rinsed in PBS once before adding primary antibody (polyclonal rabbit anti laminin-5 γ2 1:1000 or peroxidase conjugated anti actin 1:1000 in PBS supplemented with 0.1% tween-20 and 0.5% non-fat dry milk) and incubating overnight (appr. 16 hours) at 4° C. Membranes were rinsed 3×5 minutes with PBS supplemented with 0.1% tween-20, and incubated with secondary antibody conjugated with horse radish peroxidase (goat anti rabbit 1:15,000 in PBS supplemented with 0.1% tween-20 and 0.5% non fat dry milk (for detection of laminin-5 γ2)) and incubate 1 hour at 25° C. The membranes were rinsed 3×5 min in PBS supplemented with 0.1% tween-20 before visualisation with ECL (Amersham) according to supplier instructions.

In FIG. 3, from the western blot of SW480 cell lysates, the immunoblot detection of laminin-5 γ2 is shown in at the top whereas the immunoblot detection of actin is shown underneath. The immunoblot of laminin-5 γ2 show two clear bands representing the long 155 kDa and the shorter 105 kDa forms of the chain. In all the siRNA against laminin-5 γ2, the decreased expression is mainly seen on the large form of the chain. This long form of the laminin-5 γ2 is also the form secreted from the cells and is therefore expected to the one first affected by siRNA.

### Example 3

### Cytotoxicity of siRNA in Combination with Other Anti-Cancer Drugs

This example shows that susceptibility to cell death induction is increased by reducing laminin-5 γ2 expression.

SW480 cells obtained from ATCC were grown in DME:F12 supplemented with 10% foetal calf serum and routinely checked for being mycoplasma free. For transfection, the cells were seeded as 3×10⁵ cells/2 ml in 6 well plates. Transfection was carried out after cells have attached at least overnight using Lipofectamin 2000 (Life Technologies). Liposomes were formed in medium without serum by mixing 10 μl lipofectamine 2000 and 3 μg plasmid DNA or 15 μl 20 pmol/μl oligonucleotide in a total volume of 250 μl. After liposome formation, the mixture was added to cells and incubated 5 hours in cell incubator before medium was changed to complete medium with serum. After 24 hours, cells were trypsinised and moved to 96 well plates to analyse for death induction.

The cells were allowed to plate for 24 hours before dosing with the indicated compounds, all in triplicate. The compounds were solubilised in DMSO and added to cells in medium with no more than 0.1% v/v DMSO. Triplicate cell cultures for basal cell death (untreated) and maximal response (complete lysis) were included as well as triplicate media samples for background determination. Cells were incubated for additional 24 hours. The maximal response (100% cell death) was induced by adding 1% v/v Triton X100 to triplicate cell cultures 5 min before medium was collected for detection of lactate dehydrogenase (LDH) activity. Medium from all samples were used in the detection of LDH using a kit from Takara Bio Inc.

The transfection of SW480 cells with siRNA against gamma2 increased the spontaneous cell death when compared to SW480 cells transfected with scrambled RNA of the same length. Addition of low dose of thapsigargin, staurosporine, TRAIL or tunicamycin did not attenuate the increased cell death by siRNA against gamma2. The combination with any of the anti-cancer compounds was still effective for cell death in the SW 480 cells.

1. A method of treating inflammatory bowel disease comprising administering to a subject suffering from inflammatory bowel disease a composition comprising an effective amount of a laminin-5 modulator.

2. The method of claim 1, wherein the inflammatory bowel disease is ulcerative colitis.

3. The method of claim 1, wherein the inflammatory bowel disease is Crohn's disease.

4. The method of claim 1, wherein the laminin-5 modulator is selected from the group consisting of an antibody directed against laminin-5, a laminin-5 binding protein, a siRNA, an antisense molecule, or a small molecule Ln-5 modulator.

5. The method of claim 4, wherein the laminin-5 modulator is an antibody against laminin-5.

6. The method of claim 5, wherein the method comprises a step of determining that the subject comprises a number of non-mucin producing Ln-5-immunopositive cells prior to treatment.

7. A method of reducing the risk of inflammatory bowel disease-associated colorectal carcinoma comprising the step of administering to a subject suffering from inflammatory bowel disease a composition comprising an effective amount of a laminin-5 modulator.

8. The method of claim 7, wherein the inflammatory bowel disease is ulcerative colitis.

9. The method of claim 7, wherein the inflammatory bowel disease is Crohn's disease.

10. The method of claim 7, wherein the laminin-5 modulator is selected from the group consisting of an antibody directed against laminin-5, a laminin-5 binding protein, a siRNA, an antisense molecule, or a small molecule Ln-5 modulator.

11. The method of claim 10, wherein the laminin-5 modulator is an antibody against laminin-5.

12. A method of treating autosomal dominant polycystic kidney disease in a subject comprising administering to a subject in need thereof with a composition comprising an effective amount of a laminin-5 modulator.

13. The method of claim 12, wherein the laminin-5 modulator is selected from the group consisting of an antibody directed against laminin-5, a laminin-5 binding protein, a siRNA, an antisense molecule, or a small molecule Ln-5 modulator.

14. The method of claim 13, wherein the laminin-5 modulator is an antibody against laminin-5.
